SEQUENCE LISTING (4 sequences)

SEQ ID NO: 1 - DNA, 3254 bp, Artificial Sequence
  Synthetic Construct
cactgtgcacattctcctacaggattgttcccacaccaacacttgaaaaatatccaatttcaaacacagtaagctgcatcaaagaaaattatccatgagaactgctttatcttactttaatattgaaaatgaatttcaaggtaaccagaaaaaagacccacttcgacccactaatatgatttacagtaaaaatctgctcttaaaaaaattactttagaacttagagtcttaaatgtaattaacgttttgctgttatgcagtcgccaccgaaattctctccaaattcataagaaacattcaatattattaagtccaaccaaaaaaggaataacatttaaagtatcaagatgatacaggttaaatttttatataagggaaaaatgtaagaaaaatagttacaatctacataatgtatcttgaaatcgatgaaatatataaaaaggcatcctttgaaataataatggcaaatagctcctaaatattaagacacttaaagctacatgctttaatttccgaatgagcagaacgaaaatttatgtttaagctaggcaccgaaggttactgtcaggatataatcttacttataggatataatctttaataagactagggaaaacaatctttcctaagaagagcataccaaactgttttttatgtacacgaatgtcatgcagctttaacgaacgaatgcctgcgttccctggtaagaaaatgttagaaggcatatatcatttaaaagatacaggatgcaaatttaatccacatctaaaagctaaagctcggtaaggttaaaaaagtttttgcatcacttaaaacacctgtaggagaatgcggccgcgaagttcctattctctagaaagtataggaacttccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttgaagttcctattctctagaaagtataggaacttcggtaccgtgcacagtggcatattcaagtgacattttagcagaatttcataagtatgtgctacagaactgtaatatttcataataacgaaaaaatacatatttgaaacgtaatgaatcatataacaaaaatgaaattttactacccaagaaactgagtgacgctaatatttttgcagcaaagaaagaaaaaggtaaaagcaaatgtggaatagattatcttcatcaccacaccttccatcatccctcagccataatcactttggtgtaatattagggaagtgcacaaacgtcaaaatctgacagacacgcggagctgtagcccaactccacatcaaatcgctctgtctcgatggcgtttgccgttaagtgcaacgaggagaccaatctcctctgaggattgtgccacaattcacgaaggtggggcggcagcctaccggcacaaaaaaaaaaaaaaagcttaccggaggaaaaaaaagaggcggggggtggagacagagccaacagccattttccctcagagttcaaaacacaggcacttttctcgagcgccggatccgcacccattagcgtggttagagacactccacccgggtggtggtggacaaaagagggcctaacggcccccaccccaacaagagaacgaagatacacttaacaagccccagccgctgaatccccgaggaggtgcggaggggtggctactcactgtttaatgtagggccttttacctcaaaaacaaactacgcgaaagcgaaaggaaatagaaagctcaacccctactaccggaaaaaaactgccacggccgagctccctgtaggagaatgtgcacagtg

SEQ ID NO: 2 - DNA, 20 bp, Artificial Sequence
  Synthetic Construct
cactgtgcacattctcctac

SEQ ID NO: 3 - DNA, 10586 bp, Artificial Sequence
  Synthetic Construct
accaaagtgattatggctgagggattgttcccacaccaacgcttgaaaaatatccaatttcaaacacagtaagctgcatcaaaaaaaaattatccatgaaaactgctttatcttactttaatattgaaaatgcatttcaaggtaaccagaaaaaagacccacttctacccactaatatgatttacagtaaaatctgctcttaaaaaaattactttagaacttagtcttaaatgtaattaatgttttgctgttatgcagtcgccaccgaaattctctctaaattcataagaaacattcaatatgattaagtccaaccaaaaaaaggaataacatttaaaatatcaagatgatataccttaaaattttatatatgggaaaaatctaagaaaaatagttacaatctacgtaacgtatcttgaaatttatcaaatataacatataaaaaggcgtcctttgaaataattacggcaaatagctcctaaatagtaagacacttaagctacacgctttaatttccgaatgagcagaacgaaaatttaaactaggcaccgaaagttattgtcaggatataatcttacttataggatataatctttaataagactagggaaaaaaaaaaaaactttcctaagagcacaccaaactgttttttatgtacacaaatgtcatgcagctttaacgaacgaatgcctgcgttccctggtaagaaaatgttacagggcgtatatcatttaaaaaatacagaatgcaaatttaatccacatctaaaagctaaagctcaataaggtttaaaaagctcttacattacttaaaacacctgtaggagaatgtgcacagtggcatattcaagtaacattttagcagaatttcgtaagtatgtgctacagaactgtaatatttcataataacgaaaaattttcaatatttgaaacgtaatggatcacataacaaaaatgaaattgtactacttaagaaactgagtgaagctactacttctgtagcaaagaaagaaaaaggtaaaagcaaatgtggaacagattatcttcaccaccacaccttccattaaccctcagccataagcggccgcgaagttcctattctctagaaagtataggaacttctacttgaatgcagttatagaaagtaccttatctattcatcattgctatggaatacataagagtcttctatattcaaggtattttatattcaattttgacgtaaaaacaaaaaggaaaagattttaatgtttgtgttcttttgcattgctgttagtgtggatggactcggtgtccatgagcaagtataaataaaccagccggtgcaaatcttgttcttttggggcaagccttttgcattttaaaagcaacatcacatgctttgcatacctacctttccacatctttatactatcataacatggcttgggcagatgaaatccagaaagttttgactatgcttatgtattgacagtctaatattttatgtgaaaaagtaaacaaagcaggactatgtattgaatttggcagcctatttccttgttctatgaatatgcaaaaaatacagctgttcagagtttgtccacgatcccacaagaacagcactaacatactgatcggctgactgtattctaatatttccctgttaggcgtgtccacaaatcccaaaatgtgcactgagagatttgcctgctgccacaggcagccaactctgcttaagaggaaaacatcacttgactatctaagtgctttatgcttcatataaacaaacaagcaaaaaaattgtataacagaatctgtctccagacttgcagattgtcgagaaggacatgatgtctgttggggtagtcgaaaggcatggaacatatttaggtatatttactcttcttacctttcaggaaaaaccaaatgacctactgttataaagaatattgctttttattccctcagtaccaaatcaataaaagtcctttcagcacaacagctgttccttaatggccccagttaatgcactgcctgaatcaaacacattccagtgtctacaccaggcagaccctcaattaatggtgatggatcgactttctgacaagcagtagaaactcaatttatagaaattgatcctatgagacaaaaataaataatttaagagcactgtgtgttaaattctatgtttggcaacataacaatgtgtatagatctaagaactatgtgtattttttttaatgaaaaggttttccagttagctgacagatactgagtgtctcaatcattttgagttataatccaagcatagcaaaactataaatctgggtgattcatatgaatattctgatagtgatatcataatgattatgaagaatctgaagactcgtttacctaaagataagacacatgtttgttcgttagtaattataactttaaatcttgcaataaagattgcctattagtgataaacactatttgaattgtccatttcagatttttagtgtacagattttcataactttctacattattgtacactattctttaaggaagatgagcaggtttgaaagagggccagaagtaaccacttaatttgaaagtataatgaattttctctttcgctgccatagaactgtgtggttttaccctttgccaggtgacagcatctccttagtcaaagcagagtcttagaaaatgagggaatgggctgttgggtacttcagcagagcattgctcttatatcctatgtccaaaccagagcactagctgcgctctacattccaaaaagtttgagcaaataaactgacaagcatctaccactacaagatgcaaccggatcgctcctttcaatcttccgctagtcggtatagacaatccagtttcacttacgatgtcaaaagggacgtctacaacgaggaaaattttcaacaggaacatcgaaaaaagacggcctcatcagggaatgtcgatattgacattagtactgtgtcccaccacgttgagtgtcgatgttcttggtacaaattccgcaggtgtctgctcaccgtttttccgtttttggagtggatgtgtttttatagatttaaggattggctgttgggtgatttgttggccggaatatccgttggactggtccaaatccctcaagttctcatgttgggtttgttggcgcgccacttgattccacctttgaatgtgagctacgcagcattctgtgctagcgtgatttatggaatctttggatcctgccaccagatgagtataggcacctttttcctggttagcgcgctcaccattaacgtccttagaaccgaaccatttaactctggccatctcctgctgggtaccttcatccaggatgatttcagcaacataactttttacgaaaactataatcgcagtctctcaagtgtcgctagtgtgaccctgctgactgggatcatacagcttagtatgggtatgttgggctttggcttcatcgtgacatacatccccgaagcggctataagcgggtatctggcagcaacagccctgcatattatgttgtcccagcttacgtgtatattcggcgtgatgatcagctataatagtggcccgatagcttttttctacaacataatcaactactgccttggactcccaaaagctaactctacatccatactgctgtttcttacgactatagtggcgttgcggatcaataagtgtattcgcatttcatttaatcaataccccattgagtttccaatggaaatctttttgatactcggattcgctgctttctccaacaaggtcaatatggcaactgaaaattcacttatgcttatcgagatgattccctatagcttccttttccctgtgacaccggatatgtccaatctgactgaggtcttggcagagagcttctcattggccttggtctcttcatttcttttgattttcttgggcaaaaagatagccagtttccataattatgatgttaatagtaatcaagatctcattgccattggcttgtgtaatgttgttagtagcttttttaggtcatatgtcttcacgggagcagtcgcccgaacgatcattcaagataaaacaggtggccggcagcagttcgcctcccttgtgggcgcggggctcatgcttctgcttatgatgaagatggctcattttttttataagctccccaatgctatagtggctggaattattctttctaacgtgctgccttatttggaagtcgtttacacacttcctagtctgtggcgacaaaaccagtacgattgtttgatttggatggtgacctttatgagtgccatattgcttggacttgatatcgggctggtcgtggcagttaccttcgccttctttataatcactgtgcagtctcatagaaccaaaattctcctgctgggacaaattcccaacacaaatatatatcgaagctttcaagactatcgcgaagttgcgaatatcccgggggtcaagattttccagtgttgtaatgcgattacgttcgtgaatgttcattaccttaagcggaaagttcttgaagaaatagaaatggtcaaaatgccccttacagatgaagaaatctatacgctgtttagccccaatgaagaaggagcccagagaggtaaaatatgtagatgctattgtaactgtgacgagcctgagccgagcccaagagttatctataccgagcgatatgaagttcaaaggggccgggaatcttcattcatcaacttggtgagatgtagtaggtttgagtctgtcgacaccggtcaatcaatgagtgaagatcaagttccatatattacctcatcatcaagtcagagaaatccaaactacgaggaggtcgagaaagtctggctcagcgatgacccatcccggtctatgaccattactctgccggaagcttctaatacacaggtcagggccaccaaacttcttccatatagcacttcaaccgtgctgccaagtgttcacactataatcctcgatttcagcatggtgcatcttgttgatgctcaggctctggtggtccttcgacagatgttttgtgctttccagaacgtcaatatacttgtcttgatcgccggctgccattctttcgtcgtccggtccctggaaaaaaatgacttctttgatgctgggattactaaagcccagttgtttttgaccctgcacgacgcagtgctgtttgcactgtcccgaaaacttccagagtcttcagaactctctgtcgatgaaagcaagactgtcattcaggaaacgttcagcgaaacggacaaaaaagaggaatccagacataaaacatctaggagcttcattgaggcgcctcggtctaagagcccggctttttctctcctccctgacccagaaatggaagaggagtcagaccttgacttgtattccactatacagatgagcaaagaccacggccttgacctcgatctggatcttgaccgagaggtcgagccggaatccgagctggagcccgagtctgaacttgatcaagagactgagcttgagccagaccccgaagcgagccacaagcctacaaggcaaaagtactggtcattgtttcgagccatcattcccagatctcccacccatacccaagcccgcacccaaagtgtcgacaggcggcaccagaatgttaaaccgtatacatctaaggcggatacttctgaagagatcgattacaaggatcatgacggggattacaaagatcatgatatagattacaaagatgatgacgataagtagattttctctacccacagcgtttgatgtattattattattatttttgcaggcctcagtaatttgggattatgaatgggattctattttaccaaagtaattcaatttttataatcaagattctatttttgagtttcaaagagaaattatatattcttctaccaaagattgattacaagcaaggctacttagggattagttttggtttaaagagaatgaagactgaataaaataaaatcactagaaaattgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctctttcctacagctcctgggcaacgtgctggttgttgtgctgtctcatcattttggcaaagaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcgaagttcctattctctagaaagtataggaacttcggtaccataacttcgtatagcatacattatacgaagttatcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctaggagacgaccttccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcgggatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttataacttcgtatagcatacattatacgaagttatggtacctcactttggtgtagtattcgggaagtgcacaaacgtcaaaatctgacagacatgcggagctggagcccaactccacttcaaatcgctcagtctcagtgacggcgtttgccgttaagtgcaacgaggagaccaatctcctctgaggattgtgccacaattcccgaaggtggggcggttagcctcccggcacaggaaaaaaaaaaaaagcttaccggaggaaaaaaaaggcgggggggggacggggggtggagacagaaacaagagccattttcctttacagttcaaaacacaggcacttttatagagcgccggatccgcacccattagccgggttagagacactccacccgggtggtggtggacaaaagagggcctaacagcccccaccccaacaaaaggacgaagatatacttaacaagctccagccgctgaatccctgaggaggtgcggaggggtggctcctcactgtttaatgtagggccttttacctcaaaaacaaactacgcgacagcgaaaggaaatacaaagctcaacccccagtaccggaaaaaaaactgccacggccgagctccggcctgtagcctgagggaaaaaggccctgctcccaactccacacgttgcggccctcttacctcttttcttccgcgcggtggcgtttctctgtcgttagatgggctccaggtggaccgtttctcctgtttaacgtccgcttagtgctcccgcagctgcggtttcaccttttagggcctggtctcttcaccccgaatctgtcctagctgccaacaccaatactaaaatgagccgctaacgcggtagtctcccgtaaaggagaaggcaacacaagcagtcaagctcctgaaaggcctgggagctgagtgacgcttgaaggggctgggctccacaaaaccccttcagggcgggatacgaggccgctggagttccagaaatcgcttgccccggccctcttcaatatggcggattcagcaagccaaaaaaaaacggccgaaccaccggcaaccgcgacggcgcaaactcccaccagcctcagcgccagtcggagcctccaatatgacgccgcgccacgccgctccgccccctcgaagtcccccctctcgactccgcctccttatcctgcctccctcagccataatcactttggt

SEQ ID NO: 4 - DNA, 20 bp, Artificial Sequence
  Synthetic Construct
accaaagtgattatggctga

(19) United States
(12) Patent Application Publication
(10) Pub. No.: US 2023/0032810 A1
(43) Pub. Date: Feb. 2, 2023

(54) METHODS AND COMPOSITIONS FOR HIGH EFFICIENCY HOMOLOGOUS REPAIR-BASED GENE EDITING

(71) Applicant: AgGenetics, Inc., San Diego, CA (US)

(72) Inventor: James WEST, Nashville, TN (US)

(73) Assignee: AgGenetics, Inc., San Diego, CA (US)

(21) Appl. No.: 17/786,478

(22) PCT Filed: Dec. 17, 2020
(86) PCT No.: PCT/US2020/065478

§ 371 Date: Jun. 16, 2022

Related U.S. Application Data
(60) Provisional application No. 62/950,357, filed on Dec. 19, 2019.

(51) Int. Cl. C12N 15/11; C12N 15/90; C12N 15/85

(52) U.S. Cl. CPC C12N 15/11; C12N 15/907; C12N 15/8509; C12N 2310/20

(57) ABSTRACT

Provided herein are methods and compositions for high efficiency homologous repair-based gene editing. The methods and compositions of the subject invention can be useful in producing gene-edited livestock and improving human medicine.

### CROSS REFERENCE TO RELATED APPLICATIONS

This application claims priority to U.S. 62/950,357, filed Dec. 19, 2019, the disclosure of which is herein incorporated by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 186122001040SEQLIST.txt, date recorded: Dec. 7, 2020, size: 18 KB).

### FIELD

This invention relates generally to the field of gene editing. More specifically, this invention relates to homologous repair-based gene editing.

### BACKGROUND

Gene editing (also known as genome editing) is an important and useful technology in genomic research and various applications. Based on the mechanism of targeted integration, gene editing can be categorized into homologous repair-based gene editing and non-homologous repair-based gene editing. Homologous repair-based gene editing, such as homology mediated end joining (HMEJ)-based gene editing, is especially useful in precision editing and insertion of large constructs, in which a user-generated repair template is provided and used by a cell to repair the damage caused by an endonuclease via homologous recombination. These different types of gene editing can be realized by various systems, including the clustered regularly interspersed short palindromic repeats (CRISPR)-nuclease system, the transcription activator-like effector nuclease (TALEN) system, and the zinc finger nuclease (ZFN) system.

Homologous repair-based gene editing has tremendous value in many fields. It greatly simplifies gene editing in livestock and thus eliminates the need for costly and time consuming cell culture and cloning. It is also essential for a host of human medicine applications: for instance, there are a great number of human diseases for which autologous transplantation with gene editing would be transformative.

However, homologous repair-based gene editing generally has a low efficiency. By way of example, while the CRISPR system itself can have efficiencies greater than 80% with well selected guide RNAs, the current state of the field for homologous repair-based gene editing generally has efficiencies of less than 10% and usually less than 1%.

Accordingly, there exists a need for improved methods and systems for high efficiency homologous repair-based gene editing.

### BRIEF SUMMARY

To address the above and other needs, the present disclosure provides new methods and materials for high efficiency homologous repair-based genome-editing. Also provided are genome-edited animals produced by the methods of the present invention.

In one aspect, the present invention provides a method for high efficiency homologous repair based genome-editing comprising: (a) providing a cell from a bovine, an equine, a caprine, an ovine, a canine, a cervid, or a porcine animal, wherein the cell comprises a genome comprising a first genome homologous region, a second genome homologous region, and a genome cut site between the first genome homologous region and the second genome homologous region; and (b) introducing a genome-editing polypeptide that introduces at least a single stranded break at the genome cut site and a circular polynucleotide comprising a first targeting homologous region and a second targeting homologous region, wherein either (i) the circular polynucleotide comprises a new polynucleotide sequence between the first targeting homologous region and the second targeting homologous region, or (ii) the first targeting homologous region and the second targeting homologous region lack a third genome region between them that is between the first genome homologous region and the second genome homologous region, wherein the first targeting homologous region is homologous to the first genome homologous region and the second targeting homologous region is homologous to the second genome homologous region, and wherein the genome-editing polypeptide introduces a least one strand break at the genome cut site and either (1) the new polynucleotide sequence is introduced into the genome of the cell between the first genome homologous region and the second genome homologous region by homologous recombination between the first genome homologous region and the first targeting homologous region and between the second genome homologous region and the second targeting homologous region, or (2) the third genome region is deleted from the genome of the cell by homologous recombination between the first genome homologous region and the first targeting homologous region and between the second genome homologous region and the second targeting homologous region.

In some embodiments, the circular polynucleotide further comprises a first circular polynucleotide cut site 5′ to the first targeting homologous region and optionally a second circular polynucleotide cut site 3′ to the second targeting homologous region.

In some embodiments that may be combined with the preceding embodiments, the cell is an induced pluripotent stem (iPS) cell, a progenitor of a gamete, a gamete, a zygote, or a cell in an embryo.

In some embodiments, the method of the invention further comprises transferring into a suitable host female animal the zygote, the embryo, a zygote or an embryo produced from the gamete, or an embryo produced from the zygote, optionally after screening for introduction of the new polypeptide into the genome of the cell or for deletion of the third genome region from the genome of the cell.

In some embodiments that may be combined with the preceding embodiments with a first circular polynucleotide cut site and optionally a second circular polynucleotide cut site, the genome-editing polypeptide introduces at least a single stranded break at the first circular polynucleotide cut sit, the second circular polynucleotide cut site, or both.

In some embodiments that may be combined with the preceding embodiments with a first circular polynucleotide cut site and optionally a second circular polynucleotide cut site, a second genome-editing polypeptide is introduced to the cell sequentially or simultaneously with the genome-editing polypeptide and the second genome-editing polypeptide introduces at least a single stranded break at the first circular polynucleotide cut site, the second circular polynucleotide cut site, or both.

In some embodiments that may be combined with any of the preceding embodiments, the genome-editing polypeptide is a site-specific nuclease polypeptide.

In some embodiments that may be combined with any of the preceding embodiments, the genome-editing polypeptide is a TALEN polypeptide or a ZFN polypeptide.

In some embodiments that may be combined with any of the preceding embodiments, the genome-editing polypeptide is a CRISPR-nuclease polypeptide in complex with a targeting polynucleotide that hybridizes at or adjacent to the genome cut site.

In some embodiments that may be combined with any of the preceding embodiments with a CRISPR-nuclease polypeptide, the CRISPR-nuclease polypeptide is a single-strand-specific or double-strand-specific nuclease that is site-directed by a guide RNA

In some embodiments, the CRISPR-nuclease polypeptide is a Cas9 polypeptide, a Cas12 polypeptide, a Cascade polypeptide, or a CasZ polypeptide.

In some embodiments that may be combined with any of the preceding embodiments, the genome-editing polypeptide introduces a double-stranded break that is blunt or staggered.

In some embodiments that may be combined with any of the preceding embodiments, the circular polynucleotide is a vector or a plasmid.

In some embodiments that may be combined with any of the preceding embodiments, the circular polynucleotide does not comprise a bacterial origin of replication.

In some embodiments that may be combined with any of the preceding embodiments that have a new polynucleotide sequence, the new polynucleotide sequence comprises one or more point mutations.

In some embodiments that may be combined with any of the preceding embodiments that have one or more point mutations, the one or more point mutations introduce a stop codon in a polypeptide coding region at or adjacent to the genome cut site; introduce a new DNA-binding site for a transcription enhancer or a transcription repressor at or adjacent to the genome cut site; alter or eliminate a DNA-binding site for a transcription enhancer or a transcription repressor at or adjacent to the genome cut site; or change a gene at or adjacent to the genome cut site from a first allele to a second allele.

In some embodiments that may be combined with any of the preceding embodiments that have one or more point mutations, the one or more point mutations is at least 2, 3, 4, 5, 6, 7, 8, 9, 10 15, 20, 25, 30, 35, 40, or 50 insertions, deletions, substitutions, or combinations thereof.

In some embodiments that may be combined with any of the preceding embodiments that have one or more point mutations, the one or more point mutations is less than 2, 3, 4, 5, 6, 7, 8, 9, 10 15, 20, 25, 30, 35, 40, 50, 60, 70, 80, 90, or 100 insertions, deletions, substitutions, or combinations thereof.

In some embodiments that may be combined with any of the preceding embodiments that have a new polypeptide, the new polynucleotide sequence comprises a transgene.

In some embodiments that may be combined with any of the preceding embodiments that have a transgene, the transgene comprises one or more of the following: a promoter region; an enhancer region; a transcription termination regions, and a polypeptide coding region that optionally further comprises a polyadenylation site.

In some embodiments that may be combined with any of the preceding embodiments that have a new polypeptide, the new polynucleotide sequence further comprises a selectable or screenable marker optionally flanked by excision sequences.

In some embodiments that may be combined with any of the preceding embodiments that have excision sequences, the excision sequences are loxP sites or FRT sites.

In some embodiments that may be combined with any of the preceding embodiments that have a third genome region, the third genome region is less than 9000, 8000, 7000, 6000, 5,000, 4,000, 3,000, 2,500, 2,000, 1,500, 1,000, 900, 800, 700, 600, 500, 400, 300, 250, 200, 150, 100, 90, 80, 70, 60, 50, 40, 30, 25, 20, 18, 16, or 14 nucleotides long.

In some embodiments that may be combined with any of the preceding embodiments that have a third genome region, the third genome region is at least 10, 12, 14, 16, 18, 20, 25, 30, 35, 40, 45, 50, 60, 70, 80, 90, 100, 125, 150, 175, 200, 225, 250, 300, 350, 400, 500, 600, 700, 800, 900, 1,000, 1,250, 1,500, 1,750, 2,000, 2,250, 2,500, 2,750, 3,000, 3,250, 3,500, 3,750, 4,000, 4,250, 4,500, 4,750, 5,000, 6,000, 7,000, 8,000, or 9,000 nucleotides long.

In some embodiments that may be combined with any of the preceding embodiments, adjacent to the genome cut site is within 5,000, 4,000, 3,000, 2,500, 2,000, 1,500, 1,000, 900, 800, 700, 600, 500, 400, 300, 250, 200, 150, 100, 90, 80, 70, 60, 50, 40, 30, 20, 10, or 5 nucleotides of the at least single stranded break.

In some embodiments that may be combined with any of the preceding embodiments, the first genome homologous region is less than 5,000, 4,000, 3,000, 2,500, 2,000, 1,500, 1,000, 900, 800, 700, 600, 500, 400, 300, 250, 200, 150, 100, 90, 80, 70, 60, 50, 40, 30, 25, 20, 18, 16, or 14 nucleotides long.

In some embodiments that may be combined with any of the preceding embodiments, the second genome homologous region is at least 10, 12, 14, 16, 18, 20, 25, 30, 35, 40, 45, 50, 60, 70, 80, 90, 100, 125, 150, 175, 200, 225, 250, 300, 350, 400, 500, 600, 700, 800, 900, 1,000, 1,250, or 1,500 nucleotides long.

In some embodiments that may be combined with any of the preceding embodiments, the second genome homologous region is less than 5,000, 4,000, 3,000, 2,500, 2,000, 1,500, 1,000, 900, 800, 700, 600, 500, 400, 300, 250, 200, 150, 100, 90, 80, 70, 60, 50, 40, 30, 25, 20, 18, 16, or 14 nucleotides long.

In some embodiments that may be combined with any of the preceding embodiments, the first genome homologous region is at least 10, 12, 14, 16, 18, 20, 25, 30, 35, 40, 45, 50, 60, 70, 80, 90, 100, 125, 150, 175, 200, 225, 250, 300, 350, 400, 500, 600, 700, 800, 900, 1,000, 1,250, or 1,500 nucleotides long.

In some embodiments that may be combined with any of the preceding embodiments, the first genome homologous region and the second genome homologous region are on the same chromosome.

In certain embodiments that may be combined with any of the preceding embodiments, the first genome homologous region and the second genome homologous region are less than 9000, 8000, 7000, 6000, 5,000, 4,000, 3,000, 2,500, 2,000, 1,500, 1,000, 900, 800, 700, 600, 500, 400, 300, 250, 200, 150, 100, 90, 80, 70, 60, 50, 40, 30, 25, 20, 18, 16, or 14 nucleotides apart.

In some embodiments that may be combined with any of the preceding embodiments, the first genome homologous region and the second genome homologous region am at least 10, 12, 14, 16, 18, 20, 25, 30, 35, 40, 45, 50, 60, 70, 80, 90, 100, 125, 150, 175, 200, 225, 250, 300, 350, 400, 500, 600, 700, 800, 900, 1,000, 1,250, 1,500, 1,750, 2,000, 2,250, 2,500, 2,750, 3,000, 3,250, 3,500, 3,750, 4,000, 4,250, 4,500, 4,750, 5,000, 6,000, 7,000, 8,000, or 9,000 nucleotides apart.

In some embodiments that may be combined with any of the preceding embodiments, the new polynucleotide sequence is at least 10, 12, 14, 16, 18, 20, 25, 30, 35, 40, 45, 50, 60, 70, 80, 90, 100, 125, 150, 175, 200, 225, 250, 300, 350, 400, 500, 600, 700, 800, 900, 1,000, 1,250, 1,500, 1,750, 2,000, 2,250, 2,500, 2,750, 3,000, 3,250, 3,500, 3,750, 4,000, 4,250, 4,500, 4,750, 5,000, 6,000, 7,000, 8,000, 9,000, 10,000, 12,500, 15,000, or 20,000 nucleotides long.

In certain embodiments that may be combined with any of the preceding embodiments, the new polynucleotide sequence is less than 50, 60, 70, 80, 90, 100, 125, 150, 175, 200, 225, 250, 300, 350, 400, 500, 600, 700, 800, 900, 1,000, 1,250, 1,500, 1,750, 2,000, 2,250, 2,500, 2,750, 3,000, 3,250, 3,500, 3,750, 4,000, 4,250, 4,500, 4,750, 5,000, 6,000, 7,000, 8,000, 9,000, 10,000, 12,500, 15,000, 20,000, 30,000, 40,000, or 50,000 nucleotides long.

In other embodiments that may be combined with any of the preceding embodiments, the new polynucleotide sequence is between 50 and 50,000, 60 and 50,000, 70 and 50,000, 80 and 50,000, 90 and 50,000, 100 and 50,000, 125 and 50,000, 150 and 50,000, 175 and 50,000, 200 and 50,000, 225 and 50,000, 250 and 50,000, 300 and 50,000, 350 and 50,000, 400 and 50,000, 500 and 50,000, 600 and 50,000, 700 and 50,000, 800 and 50,000, 900 and 50,000, 1,000 and 50,000, 1,250 and 50,000, 1,500 and 50,000, 1,750 and 50,000, 2,000 and 50,000, 2,250 and 50,000, 2,500 and 50,000, 2,750, 3,000 and 50,000, 3,250 and 50,000, 3,500 and 50,000, 3,750 and 50,000, 4,000 and 50,000, 4,250 and 50,000, 4,500 and 50,000, 4,750 and 50,000, 5,000 and 50,000, 6,000 and 50,000, 7,000 and 50,000, 8,000 and 50,000, 9,000 and 50,000, 10,000 and 50,000, 12,500 and 50,000, 15,000 and 50,000, or 20,000 and 50,000 nucleotides long.

Another aspect of the invention includes compositions comprising the genome-editing polypeptide, the circular polynucleotide, and optionally the cell of the preceding aspect in any and all of its embodiments.

In yet another aspect, the present invention provides a genome-edited animal produced by any one of the preceding methods and any and all of the various embodiments.

In some embodiments, the present invention provides a genome-editing kit comprising the genome-editing polypeptide, the circular polynucleotide, and optionally the cell of any one of the preceding methods and any and all of the various embodiments.

### BRIEF DESCRIPTION OF DRAWINGS

FIG. 1 shows the structure of the genetic construct used in high efficiency homologous repair-based gene editing targeting cattle Y chromosome. The construct was assembled in a pUC57 backbone, and was injected as a circular plasmid. Nucleotide sequence of this genetic construct is shown in SEQ ID NO. 1. The target sequence is shown in SEQ ID NO. 2.

FIGS. 2A and 2B show high efficiency homologous repair-based gene editing in cattle oocytes as indicated by expression of the green fluorescent protein (GFP). FIG. 2A shows cattle oocytes injected with ultra-pure water as control and FIG. 2B shows cattle oocytes injected with a mixture of Cas9 protein, single guide RNA (sgRNA), and the genetic construct of FIG. 1.

FIGS. 3A-3C show the three categories of cattle embryos developed from the edited cattle oocytes in FIG. 2B. Specifically, FIG. 3A shows an exemplary cattle embryo with no GFP expression; FIG. 3B shows an exemplary cattle embryo with expressed GFP in some, but not all, cells in the embryo; and FIG. 3C shows an exemplary cattle embryo with expressed GFP in apparently every cell in the embryo.

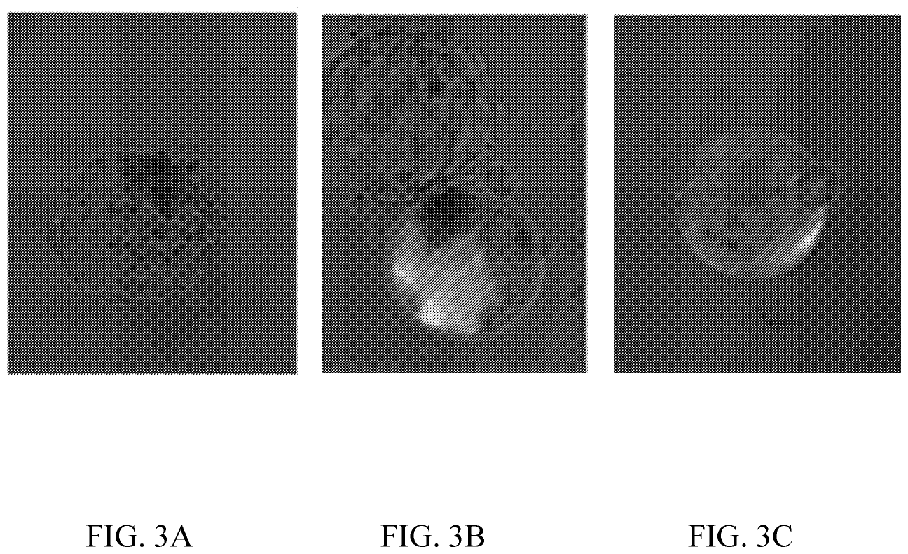

FIG. 4 shows the structure of the genetic construct used in high efficiency homologous repair-based gene editing targeting goat Y chromosome. Nucleotide sequence of this genetic construct is shown in SEQ ID NO. 3. The target sequence is shown in SEQ ID NO. 4.

### BRIEF DESCRIPTION OF SEQUENCES

SEQ ID NO. 1 shows the nucleotide sequence of a genetic construct for use in high efficiency homologous repair-based gene editing in cattle. Sequentially, the genetic construct comprises the following elements from 5′ to 3′: sgRNA target site, left homology arm (match to cattle Y chromosome). FRT site, CMV enhancer, CMV promoter, Kozak sequence, GFP. SV40 poly(A) signal, FRT site, right homology arm (match to cattle Y chromosome), and sgRNA target site.

SEQ ID NO. 2 shows the nucleotide sequence of the CRISPR sgRNA target site on cattle Y chromosome.

SEQ ID NO. 3 shows the nucleotide sequence of a genetic construct for use in high efficiency homologous repair-based gene editing in goats. Sequentially, the genetic construct comprises the following elements from 5′ to 3′: sgRNA target site, left homology arm (match to goat Y chromosome), goat Gnat3 promoter and tether, codon-optimized goat dnSlc26a8, goat Spam1 3′UTR, beta globin poly(A) and intron, LoxP. CMV promoter, GFP, linker, puromycin resistance, short SV40 poly(A) signal, LoxP, right homology arm (match to goat Y chromosome), and sgRNA target site.

SEQ ID NO. 4 shows the nucleotide sequence of the CRISPR sgRNA target site on goat Y chromosome.

### DETAILED DESCRIPTION

The following description sets forth exemplary compositions, systems, methods, parameters and the like. It should be recognized, however, that such description is not intended as a limitation on the scope of the present disclosure but is instead provided as a description of exemplary embodiments.

Provided herein are methods for high efficiency homologous repair based genome-editing. Also provided are compositions and kits for high efficiency homologous repair based genome-editing. Further provided are genome-edited animals produced by the methods in the present disclosure.

The methods described herein, and the compositions, kits, and other materials disclosed herein are based in part on the surprising discovery that it is possible to achieve high efficiency of genome-editing via methods that may include introducing into a cell a genome-editing nuclease polypeptide and a circular polynucleotide having cut sites of the nuclease. Genome Editing

Accordingly, in one aspect, the present disclosure provides a method for high efficiency homologous repair based genome-editing comprising: (a) providing a cell from a bovine, an equine, a caprine, an ovine, a canine, a cervid, or a porcine animal, wherein the cell comprises a genome comprising a first genome homologous region, a second genome homologous region, and a genome cut site between the first genome homologous region and the second genome homologous region; and (b) introducing a genome-editing polypeptide that introduces at least a single stranded break at the genome cut site and a circular polynucleotide comprising a first targeting homologous region and a second targeting homologous region, wherein either (i) the circular polynucleotide comprises a new polynucleotide sequence between the first targeting homologous region and the second targeting homologous region, or (ii) the first targeting homologous region and the second targeting homologous region lack a third genome region between them that is between the first genome homologous region and the second genome homologous region, wherein the first targeting homologous region is homologous to the first genome homologous region and the second targeting homologous region is homologous to the second genome homologous region, and wherein the genome-editing polypeptide introduces a least one strand break at the genome cut site and either (1) the new polynucleotide sequence is introduced into the genome of the cell between the first genome homologous region and the second genome homologous region by homologous recombination between the first genome homologous region and the first targeting homologous region and between the second genome homologous region and the second targeting homologous region, or (2) the third genome region is deleted from the genome of the cell by homologous recombination between the first genome homologous region and the first targeting homologous region and between the second genome homologous region and the second targeting homologous region.

Genome-editing, also known as genome editing or gene editing/gene-editing, is a way of making specific changes to the genomic DNA of a cell. Generally, an engineered nuclease cuts the DNA at a specific sequence, and when this is repaired by the cell's natural DNA repair machinery, a change or ‘edit’ can made to the sequence. Genome-editing can thus be used to add, remove, or alter DNA in the genome, and as a result, change the characteristics of a cell or an organism.

The high-efficiency genome-editing methods of the present disclosure may be achieved by various genome-editing systems and techniques known in the art.

Based on the DNA repair pathway that is harnessed, genome-editing can also be categorized into different types, including, for example, non-homologous end joining (NHEJ) and homology directed repair (HDR).

Non-homologous end joining (NHEJ) is the predominant cellular repair pathway that mends double-strand breaks (DSBs) in most eukaryotes. It occurs during all phases of the cell cycle and is often regarded as a “quick fix” mechanism. In general, the mechanism works by rejoining the blunt ends of DNA back together with minor processing. The pathway involves several key proteins, including Ku, DNA-PKes, and DNA Ligase 4. However, NHEJ is a relatively error-prone process, with occasional insertions or deletions (indels) left at the cut site after DNA repair when compared to the wild-type sequence.

Homology-directed repair (HDR, also known as homologous repair) is the second most common DNA repair mechanism in most eukaryotes. Unlike NHEJ, HDR relies on a homologous repair template (e.g. a sister chromatid, or an exogenous nucleic acid molecule) to repair the broken DNA. Unlike NHEJ, HDR relies on a process of homologous recombination where a DNA template is used to provide the homology necessary for precise repair of DNA breaks. This DNA template can come from within the cell during the late stage of S phase and from the G2 phase of the cell cycle, when sister chromatids are available prior to the completion of mitosis. Additionally, exogenous repair templates can be delivered into a cell to generate a precise change in the genome. As a result, with HDR. DNA is often repaired faithfully with no indel formation. However, genome-editing using HDR is generally inefficient as it is restricted to the late S/G2 phase of the cell cycle. The current state of the field for homologous repair is that genome editing efficiencies are less than 10%, and usually less than 1%.

Recently, research has shown that a new type of DNA repair system homology-mediated end-joining (HMEJ) may be active during G1/early S phases and single-strand annealing may be involved in this pathway (Yao et al. Cell Research (2017) 27, 801-814).

The cells in the methods of the subject invention can include any suitable cells including, but not limited to, induced pluripotent stem (iPS) cells, progenitor cells of a gamete, gametes, zygotes, or cells in an embryo. In some embodiments of the present invention, the cell is an induced pluripotent stem (iPS) cell, a progenitor of a gamete, a gamete, a zygote, or a cell in an embryo.

### Genome-Editing Polypeptide

In certain aspects of the present invention, the genome-editing polypeptide is a site-specific nuclease polypeptide.

Site-specific nucleases can permit the generation of single- or double-strand breaks at pre-determined positions in a genome. The creation of such breaks by site-specific nucleases prompts the endogenous cellular repair machinery to be repurposed in order to insert, delete or modify DNA at desired positions in the genome of interest. Targeted DNA cleavage mediated by site-specific nucleases is therefore an important basic research tool which has facilitated the functional determination and annotation of specific genes but amongst other things has also enabled the targeted mutation, addition, replacement or modification of genes in organisms of agricultural, industrial, or medicinal significance. During the past decades, a range of molecular tools have been developed to allow for specific genetic engineering in general, and for dedicated editing of eukaryotic genomes in particular. Initially, zinc finger nucleases (ZFN) were developed, followed by transcription activator-like effector nucleases (TALEN). Recently, a revolution has been caused by the development of the CRISPR-associated nucleases (Cas), as a more efficient, generic and cost-effective alternative for genome editing in a range of eukaryotic organisms from yeast and plant to zebrafish and human (reviewed by Van der Oost 2013, Science 339: 768-770, and Charpentier and Doudna, 2013, Nature 495: 50-51).

Talen

Accordingly, in some embodiments, the genome-editing polypeptide of the present invention is a TALEN polypeptide.

Transcription activator-like effector nucleases (TALEN) are restriction enzymes that can be engineered to cut specific sequences of DNA. They are made by fusing a TAL effector (TALE) DNA-binding domain to a DNA cleavage domain. Transcription activator-like effectors (TALEs) can be engineered to bind to practically any desired DNA sequence, so when combined with a nuclease, DNA can be cut at specific locations.

Transcription activator-like effectors (TALEs) represent a class of DNA binding proteins secreted by plant-pathogenic bacteria of the genera, such as Xanthomonas and Ralstonia, via their type III secretion system upon infection of plant cells. Natural TALEs specifically have been shown to bind to plant promoter sequences thereby modulating gene expression and activating effector-specific host genes to facilitate bacterial propagation (Römer. P., et al., Plant pathogen recognition mediated by promoter activation of the pepper Bs3 resistance gene. Science 318, 645-648 (2007); Boch. J. & Bonas. U. Xanthomonas AvrBs3 family-type III effectors: discovery and function. Annu. Rev. Phytopathol. 48, 419-436 (2010); Kay. S., et al. U. A bacterial effector acts as a plant transcription factor and induces a cell size regulator. Science 31N, 648-651 (2007); Kay. S. & Bonas, U. How Xanthomonas type III effectors manipulate the host plant. Curr. Opin. Microbiol, 12, 37-43 (2009)).

Natural TALEs are generally characterized by a central repeat domain and a carboxyl-terminal nuclear localization signal sequence (NLS) and a transcriptional activation domain (AD). The central repeat domain typically consists of a variable amount of between 1.5 and 33.5 amino acid repeats that are usually 33-35 residues in length except for a generally shorter carboxyl-terminal repeat referred to as half-repeat. The repeats are mostly identical but differ in certain hypervariable residues. DNA recognition specificity of TAL effectors is mediated by hypervariable residues typically at positions 12 and 13 of each repeat-the so-called repeat variable di-residue (RVD) wherein each RVD targets a specific nucleotide in a given DNA sequence. Thus, the sequential order of repeats in a TAL protein tends to correlate with a defined linear order of nucleotides in a given DNA sequence. The underlying RVD code of some naturally occurring TAL effectors has been identified, allowing prediction of the sequential repeat order required to bind to a given DNA sequence (Boch, J. et al. Breaking the code of DNA binding specificity of TAL-type III effectors. Science 326, 1509-1512 (2009); Moscou, M. J. & Bogdanove, A. J. A simple cipher governs DNA recognition by TALEs. Science 326, 1501 (2009)). Further, TAL effectors generated with new repeat combinations have been shown to bind to target sequences predicted by this code. It has been shown that the target DNA sequence generally start with a 5′ thymine base to be recognized by the TAL protein.

The modular structure of TALs allows for combination of the DNA binding domain with effector molecules such as nucleases. In particular, TAL effector nucleases allow for the development of new genome engineering tools known.

TAL effectors used in the practice of the invention may generate DS breaks or may have a combined action for the generation of DS breaks. For example, TAL-FokI nuclease fusions can be designed to bind at or near a target locus and form double-stranded nucleic acid cutting activity by the association of two FokI domains.

As used herein, the term “transcription activator-like effectors (TALEs)” refers to proteins composed of more than one TAL repeat and is capable of binding to nucleic acid in a sequence specific manner. In many instances. TAL effectors will contain at least six (e.g., at least 8, at least 10, at least 12, at least 15, at least 17, from about 6 to about 25, from about 6 to about 35, from about 8 to about 25, from about 10 to about 25, from about 12 to about 25, from about 8 to about 22, from about 10 to about 22, from about 12 to about 22, from about 6 to about 20, from about 8 to about 20, from about 10 to about 22, from about 12 to about 20, from about 6 to about 18, from about 10 to about 18, from about 12 to about 18, etc.) TAL repeats. In some instances, a TAL effector may contain 18 or 24 or 17.5 or 23.5 TAL nucleic acid binding cassettes. In additional instances, a TAL effector may contain 15.5, 16.5, 18.5, 19.5, 20.5, 21.5, 22.5 or 24.5 TAL nucleic acid binding cassettes. TAL effectors will generally have at least one polypeptide region which flanks the region containing the TAL repeats. In many instances, flanking regions will be present at both the amino and carboxyl termini of the TAL repeats. Exemplary TALs are set out m U.S. Pat. Publ. No. 2013/0274129 A1 and may be modified forms on naturally occurring proteins found in bacteria of the genera Burkholderia, Xanthamonas and Ralstonia.

In some embodiments, the TALEN polypeptide of the present invention may contain a nuclear localization signal (NLS) that facilitates its transportation to the nucleus.

ZFN

In some embodiments, the genome-editing polypeptide of the present invention is a ZFN polypeptide.

Zinc-finger nucleases (ZFNs) are chimeric proteins consisting of a zinc finger DNA-binding domain and a nuclease domain.

The individual DNA binding domains are typically referred to as “fingers,” such that a zinc finger protein or polypeptide has at least one finger, more typically two fingers, or three fingers, or even four or five fingers, to at least six or more fingers. In some aspect, ZFNs will contain three or four zinc fingers. Each finger typically binds from two to four base pairs of DNA. Each finger usually comprises an about 30 amino acids zinc-chelating. DNA-binding region (see, e.g., U.S. Pat. Publ. No. 2012/0329067 A1, the disclosure of which is incorporated herein by reference).

One example of a nuclease domain is the non-specific cleavage domain from the type IIS restriction endonuclease FokI (Kim. Y G; Cha. J., Chandrasegaran, S. Hybrid restriction enzymes: zinc finger fusions to Fok I cleavage domain Proc. Natl. Acad. Sci. USA. 1996 Feb. 6; 93(3):1156-60). A pair of the nuclease domain is generally required to allow for dimerization of the domain and cleavage of a non-palindromic target sequence from opposite strands.

In some embodiments, the ZFN polypeptide of the present invention may contain a nuclear localization signal (NLS) that facilitates its transportation to the nucleus.

CRISPR-Nuclease

In some embodiments, the genome-editing polypeptide of the present invention is a CRISPR-nuclease polypeptide.

In some embodiments, the genome-editing polypeptide is a CRISPR-nuclease polypeptide in complex with a targeting polynucleotide that hybridizes at or adjacent to the genome cut site.

In some embodiments, adjacent to the genome cut site is within 5,000, 4,000, 3,000, 2,500, 2,000, 1,500, 1,000, 900, 800, 700, 600, 500, 400, 300, 250, 200, 150, 100, 90, 80, 70, 60, 50, 40, 30, 20, 10, or 5 nucleotides of the at least single stranded break.

Most of the prokaryotes like bacteria and archaea makes the use of their adaptive immune system using CRISPR (clustered regularly interspaced short palindromic repeats) and Cas enzyme to detect and remove the foreign genetic material. When prokaryotes are infected by bacteriophages, then the phage DNA give rise to short cluster repeats (i.e. CRISPR) which are used to detect and cleave the DNA fragments from similar type of phages. This defense mechanism of prokaryotes is harnessed and used as a genome-editing technique.

A CRISPR-nuclease (e.g. CRISPR-associated protein 9 or Cas9) is a nuclease that uses CRISPR sequences as a guide to recognize and cleave specific strands of DNA that am complementary to the CRISPR sequence. A CRISPR-nuclease (c g. Cas9) together with a targeting polynucleotide (e.g. CRISPR sequence) can form a complex that can be used to edit a genome of interest. An example of a CRISPR complex is a wild-type Cas9 (sometimes referred to as Csn1) protein that is bound to a guide RNA specific for a target locus. As used herein the term “CRISPR-nuclease” refers to a nuclease comprising a nucleic acid (e.g., RNA) binding domain nucleic acid and an effector domain (e.g., Cas9, such as Streptococcus pyogenes Cas9). CRISPR-nucleases can also comprise nuclease domains (i.e., DNase or RNase domains), additional DNA binding domains, helicase domains, protein-protein interaction domains, dimerization domains, as well as other domains.

Accordingly, in some embodiments, the targeting polynucleotide is a guide RNA (gRNA). In some embodiments, the targeting polynucleotide is a CRISPR RNA (crRNA). In some embodiments, the CRISPR-nuclease polypeptide is a single-strand-specific or double-strand-specific nuclease that is site-directed by a guide RNA (gRNA).

A guide RNA (gRNA) is a specific RNA sequence that recognizes the target DNA region of interest and directs the Cas nuclease there for editing. The gRNA may be made up of two parts: CRISPR RNA (crRNA), a 17-20 nucleotide sequence complementary to the target DNA, and a trans-acting CRISPR RNA (tracrRNA), which serves as a binding scaffold for the Cas nuclease. While crRNAs and tracrRNAs exist as two separate RNA molecules in nature, these two RNA sequences can be artificially combined into a single guide RNA (sgRNA).

In some embodiments, the CRISPR-nuclease polypeptide of the present invention is a Cas9 polypeptide, a Cas12 polypeptide, a Cascade polypeptide, or a CasZ polypeptide. Various CRISPR systems may be used in the practice of the invention. These systems will generally have the functional activities of a being able to form complex comprising a CRISPR-nuclease and a compatible CRISPR sequence where die complex recognizes a sequence in the genome of interest. CRISPR systems can be a type I, a type II, or a type III system Non-limiting examples of suitable CRISPR proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966.

In some embodiments, the CRISPR-nuclease (e.g., Cas9) is derived from a type II CRISPR system. In specific embodiments, the CRISPR system is designed to acts as an oligonucleotide (e.g., DNA or RNA)-guided endonuclease derived from a Cas9 protein. The Cas9 protein for this and other functions set out herein can be from Streptococcus pyogenes, Streptococcus thermophilus, Streptococcus sp., Nocardiopsis dassonvillei, Streptomyces pristinaespiralis, Streptomyces viridochromogenes, Streptomyces viridochromogenes, Streptosporangium roseum, Streptosporangium roseum, AlicyclobacHlus acidocaldarius. Bacillus pseudomycoides, Bacillus, selenitireducens, Exiguobacterium sibiricum, Lactobacillus delbrueckii, Lactobacillus salivarius. Microscilla marina, Burkholderiales bacterium, Polaromonas naphthalenivorans, Polaromonas sp., Crocosphaera watsonii. Cyanothece sp., Microcystis aeruginosa, Synechococcus sp., Acetohalobium arabalicum. Ammonifex degensii, Caldicelulosiruptor becscii, Candidatus Desulforudis, Clostridium botulinum, Closiridium difficile, Finegoldia magna, Natranaerobius thermophilus, Pelotomaculumthermopropionicum, Acidithiobacillus caldus, Acidithiobacillus ferrooxidans, Allochromarium vinosum, Marinobacter sp., Nitrosococcus halophilus. Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis. Arthrospira sp., Lyngbya sp., Microcoleus chthonoplastes, Oscillatoria sp., Petrotoga mobilis, Thermosipho africanus, or Acaryochloris marina.

In some embodiments, the genome-editing polypeptide of the present invention introduces a double-stranded break that is blunt or staggered. Different genome-editing systems may have different characteristics. By way of example, Cas12a shows several key differences from Cas9 including: causing a‘staggered’ cut in double stranded DNA as opposed to the ‘blunt’ cut produced by Cas9, relying on a‘T rich’ PAM (providing alternative targeting sites to Cas9) and requiring only a CRISPR RNA (crRNA) for successful targeting. By contrast, Cas9 requires both crRNA and a transactivating crRNA (tracrRNA). A skilled artisan may determine the type of CRISPR system to practice this invention by choosing a system that has the desired characteristics that meet their needs.

In some embodiments, the CRISPR-nuclease polypeptide of the present invention may contain a nuclear localization signal (NLS) that facilitates its transportation to the nucleus.

In some embodiments that may be combined with any of the preceding embodiments, the genome-editing polypeptide introduces at least a single stranded break at the first circular polynucleotide cut site. In some embodiments, the genome-editing polypeptide introduces at least a single stranded break at the second circular polynucleotide cut site. In some embodiments, the genome-editing polypeptide introduces at least a single stranded break at both the first and the second circular polynucleotide cut site.

In some embodiments that may be combined with any of the preceding embodiments, a second genome-editing polypeptide is introduced to the cell sequentially or simultaneously with the genome-editing polypeptide and the second genome-editing polypeptide introduces at least a single stranded break at the first circular polynucleotide cut site, the second circular polynucleotide cut site, or both.

In some embodiments, the genome-editing polypeptide is a CRISPR-nuclease polypeptide and the targeting polynucleotide form a ribonucleoprotein (RNP) complex. RNPs may be assembled in vitro and can be delivered directly to cells using standard electroporation or transfection techniques. By way of example, Cas9 RNPs consist of purified Cas9 protein in complex with a gRNA. CRISPR-nuclease RNPs differ from plasmid or viral-based delivery of CRISPR components with regards to how quickly the components are expressed and how long they are present within the cell. Plasmid or viral delivery of CRISPR-nuclease and gRNA(s) requires the use of cellular transcription/translation machinery to generate functional CRISPR-nuclease-gRNA complexes, which results in a significant lag in peak CRISPR-nuclease protein expression (>12 hours). Expression of each component continues indefinitely (for lentiviral-mediated delivery) or until the DNA is lost through cell division (for plasmid or AAV-based delivery). By contrast, CRISPR-nuclease RNPs are delivered as intact complexes, are detectable at high levels shortly after transfection, and are quickly cleared from the cell via protein degradation pathways.

### Circular Polynucleotide

Accordingly, in certain aspects of the methods of the present invention, homologous repair is enabled by the presence of the first targeting homologous region and the second targeting homologous region on the circular polynucleotide, wherein the first targeting homologous region is homologous to the first genome homologous region and the second targeting homologous region is homologous to the second genome homologous region.

In certain aspects, the circular polynucleotide contains flanking nucleic sequences that direct site-specific homologous recombination/repair. In some embodiments, the circular polynucleotide comprises a new polynucleotide sequence between the first targeting homologous region and the second targeting homologous region. In some embodiments, the first targeting homologous region and the second targeting homologous region lack a third genome region between them that is between the first genome homologous region and the second genome homologous region.

The use of flanking (5′ and 3′) homologous polynucleotide sequences to permit homologous recombination into a desired genetic locus is known in the art. At present, it is preferred that up to several kilobases or more of flanking DNA corresponding to the chromosomal insertion site be present in the vector on both sides of the encoding sequence (or any other sequence of this invention to be inserted into a chromosomal location by homologous recombination) to assure precise replacement of chromosomal sequences with the exogenous DNA.

The higher the amount of sequence identity the targeting homologous regions on the circular polynucleotide share with the genome homologous regions, typically the higher the homologous recombination efficiency, and as a result, higher genome-editing efficiency. High levels of sequence identity are especially desired when the homologous regions are fairly short (e.g., 50 bases). Typically, the amount of sequencer identity between the target locus and the homologous regions will be greater than 90% (e.g., from about 90% to about 100%, from about 90% to about 99%, from about 90% to about 98%, from about 95% to about 100%, from about 95% to about 99%, from about 95% to about 98%, from about 97% to about 100%, etc.).

As used herein, “percentage of sequence identity” means the value determined by comparing two optimally aligned nucleotide sequences over a comparison window, wherein the portion of the nucleotide sequence in the comparison window may comprise additions or deletions (i.e., sequence alignment gaps) as compared to the reference sequence (which does not comprise additions or deletions) for optimal alignment of the two sequences. In other words, sequence alignment gaps are removed for quantification purposes. The percentage of sequence identity is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity One method for determining sequence identity values is through the use of the BLAST 2.0 suite of programs using default parameters (Altschul et al., Nucleic Acids Res. 25:3389-3402 (1997)). Software for performing BLAST analyses is publicly available, e.g., through the National Center for Biotechnology-Information.

The skilled artisan can determine, based on the size and characteristics of the genomic insertion or deletion, the length and content of the homologous regions used for homologous recombination and insertion of the construct at or near the location of the genome cut site. Thus, based on e.g., the teachings of U.S. Pat. No. 9,670,458, which is incorporated by reference, will readily recognize how to design the targeting homologous regions of the present invention.

By way of example, each flanking homologous region (arm) may be from a low of about 500 base pairs (bp), about 600 bp, or about 750 bp to a high of about 2 kilo base pairs (kb), about 3 kb, or about 5kb. For example, each homologous arm can be from about 500 bp to about 1 kb, from about 500 bp to about 1.5 kb, from about 500 bp to about 2 kb, from about 500 bp to about 2.5 kb, from about 500 bp to about 3 kb, from about 500 bp to about 3.5 kb, from about 500 bp to about 4 kb, from about 500 bp to about 4.5 kb, from about 500 bp to about 5 kb, from about 600 bp to about 1.5 kb, from about 600 bp to about 2 kb, from about 600 bp to about 2.5 kb, from about 600 bp to about 3 kb, from about 5600 bp to about 3.5 kb, from about 600 np to about 4 kb, from about 600 bp to about 4.5 kb, from about 600 bp to about 5 kb, from about 750 bp to about 1.5 kb, from about 750 bp to about 2 kb, from about 750 bp to about 2.5 kb, from about 750 bp to about 3 kb, from about 750 bp to about 3.5 kb, from about 750 bp to about 4 kb, from about 750 bp to about 4.5 kb, from about 750 bp to about 5 kb.

In some embodiments, the first genome homologous region is less than 5,000, 4,000, 3,000, 2,500, 2,000, 1,500, 1,000, 900, 800, 700, 600, 500, 400, 300, 250, 200, 150, 100, 90, 80, 70, 60, 50, 40, 30, 25, 20, 18, 16, or 14 nucleotides long

In some embodiments, the second genome homologous region is at least 10, 12, 14, 16, 18, 20, 25, 30, 35, 40, 45, 50, 60, 70, 80, 90, 100, 125, 150, 175, 200, 225, 250, 300, 350, 400, 500, 600, 700, 800, 900, 1,000, 1,250, or 1,500 nucleotides long.

In some embodiments, the second genome homologous region is less than 5,000, 4,000, 3,000, 2,500, 2,000, 1,500, 1,000, 900, 800, 700, 600, 500, 400, 300, 250, 200, 150, 100, 90, 80, 70, 60, 50, 40, 30, 25, 20, 18, 16, or 14 nucleotides long.

In some embodiments, the first genome homologous region is at least 10, 12, 14, 16, 18, 20, 25, 30, 35, 40, 45, 50, 60, 70, 80, 90, 100, 125, 150, 175, 200, 225, 250, 300, 350, 400, 500, 600, 700, 800, 900, 1,000, 1,250, or 1,500 nucleotides long.

In some embodiments, the first genome homologous region and the second genome homologous region are on the same chromosome.

In certain embodiments, the first genome homologous region and the second genome homologous region are less than 9000, 8000, 7000, 6000, 5,000, 4,000, 3,000, 2,500, 2,000, 1,500, 1,000, 900, 800, 700, 600, 500, 400, 300, 250, 200, 150, 100, 90, 80, 70, 60, 50, 40, 30, 25, 20, 18, 16, or 14 nucleotides apart.

In some embodiments, the first genome homologous region and the second genome homologous region are at least 10, 12, 14, 16, 18, 20, 25, 30, 35, 40, 45, 50, 60, 70, 80, 90, 100, 125, 150, 175, 200, 225, 250, 300, 350, 400, 500, 600, 700, 800, 900, 1,000, 1,250, 1,500, 1,750, 2,000, 2,250, 2,500, 2,750, 3,000, 3,250, 3,500, 3,750, 4,000, 4,250, 4,500, 4,750, 5,000, 6,000, 7,000, 8,000, or 9,000 nucleotides apart.

In some embodiments, the new polynucleotide sequence is at least 10, 12, 14, 16, 18, 20, 25, 30, 35, 40, 45, 50, 60, 70, 80, 90, 100, 125, 150, 175, 200, 225, 250, 300, 350, 400, 500, 600, 700, 800, 900, 1,000, 1,250, 1,500, 1,750, 2,000, 2,250, 2,500, 2,750, 3,000, 3,250, 3,500, 3,750, 4,000, 4,250, 4,500, 4,750, 5,000, 6,000, 7,000, 8,000, 9,000, 10,000, 12,500, 15,000, or 20,000 nucleotides long.

In certain embodiments, the new polynucleotide sequence is less than 50, 60, 70, 80, 90, 100, 125, 150, 175, 200, 225, 250, 300, 350, 400, 500, 600, 700, 800, 900, 1,000, 1,250 1,500, 1,750, 2,000, 2,250, 2,500, 2,750, 3,000, 3,250, 3,500, 3,750, 4,000, 4,250, 4,500, 4,750, 5,000, 6,000, 7,000, 8,000, 9,000, 10,000, 12,500, 15,000, 20,000, 30,000, 40,000, or 50,000 nucleotides long.

In other embodiments, the new polynucleotide sequence is between 50 and 50,000, 60 and 50,000, 70 and 50,000, 80 and 50,000, 90 and 50,000, 100 and 50,000, 125 and 50,000, 150 and 50,000, 175 and 50,000, 200 and 50,000, 225 and 50,000, 250 and 50,000, 300 and 50,000, 350 and 50,000, 400 and 50,000, 500 and 50,000, 600 and 50,000, 700 and 50,000, 800 and 50,000, 900 and 50,000, 1,000 and 50,000, 1,250 and 50,000, 1,500 and 50,000, 1,750 and 50,000, 2,000 and 50,000, 2,250 and 50,000, 2,500 and 50,000, 2,750, 3,000 and 50,000, 3,250 and 50,000, 3,500 and 50,000, 3,750 and 50,000, 4,000 and 50,000, 4,250 and 50,000, 4,500 and 50,000, 4,750 and 50,000, 5,000 and 50,000, 6,000 and 50,000, 7,000 and 50,000, 8,000 and 50,000, 9,000 and 50,000, 10,000 and 50,000, 12,500 and 50,000, 15,000 and 50,000, or 20,000 and 50,000 nucleotides long.

In some embodiments, the cell may contain multiple copies of the circular polynucleotide of invention.

In some embodiments, the circular polynucleotide further comprises a first circular polynucleotide cut site 5′ to the first targeting homologous region and optionally a second circular polynucleotide cut site 3′ to the second targeting homologous region. In some embodiments, the first circular polynucleotide cut site and the second circular polynucleotide cut site have the same nucleotide sequence of the genome cut site as recognized by the genome-editing polypeptide. Without wishing to be bound to any theory, it is postulated that the presence of the cut sites flanking the targeting homologous regions on the circular polynucleotide increases the efficiency of homologous repair and as a result the efficiency of genome-editing.

In some embodiments of the present invention, the circular polynucleotide is a vector or a plasmid.

In some embodiments, the circular polynucleotide does not comprise a bacterial origin of replication.

Genomic Insertion

In some aspects, the present invention may be useful in inserting into a genome a sequence of interest. Accordingly, in some embodiments, the circular polynucleotide comprises between the first targeting homologous region and the second targeting homologous region a new polynucleotide sequence, which is inserted at or adjacent to the genome cut site in between the first genome homologous region and the second genome homologous region in the genome.

The new polynucleotide for insertion may be of a variety of lengths, depending upon the application that it is intended for. In some embodiments, the new polynucleotide be from about 1 to about 4,000 bases in length (e.g., from about 1 to 3,000, from about 1 to 2,000, from about 1 to 1,500, from about 1 to 1,000, from about 2 to 1,000, from about 3 to 1,000, from about 5 to 1,000, from about 10 to 1,000, from about 10 to 400, from about 10 to 50, from about 15 to 65, from about 2 to 15, etc. bases).

In some embodiments of the present invention, the new polynucleotide sequence comprises one or more point mutations.

In some embodiments, the one or more point mutations introduce a stop codon in a polypeptide coding region at or adjacent to the genome cut site; introduce a new DNA-binding site for a transcription enhancer or a transcription repressor at or adjacent to the genome cut site; alter or eliminate a DNA-binding site for a transcription enhancer or a transcription repressor at or adjacent to the genome cut site; or change a gene at or adjacent to the genome cut site from a first allele to a second allele.

In some embodiments, the one or more point mutations is at least 2, 3, 4, 5, 6, 7, 8, 9, 10 15, 20, 25, 30, 35, 40, or 50 insertions, deletions, substitutions, or combinations thereof.

In some embodiments, the one or more point mutations is less than 2, 3, 4, 5, 6, 7, 8, 9, 10 15, 20, 25, 30, 35, 40, 50, 60, 70, 80, 90, or 100 insertions, deletions, substitutions, or combinations thereof.

In some embodiments, the new polynucleotide sequence comprises a transgene. In some embodiments, the transgene comprises one or more of the following: a promoter region; an enhancer region; a transcription termination regions, and a polypeptide coding region that optionally further comprises a polyadenylation site.

In some embodiments, the promoter of the transgene is a spatially or temporally specific promoter, such that it only drives expression of the transgene in certain cells or at certain developmental stages. It is within the purview of the skilled artisan to determine experimentally the optimal promoter to be used to practice the methods of the subject invention based on the teachings of the instant application and the disclosed requirements for promoter functionality for a specific physiological process.

In some embodiments, the promoter is a universal promoter. In some embodiments, the universal promoters useful in such embodiment of the subject invention include, but not limited to, cytomegalovirus (CMV) promoter, CMV-chicken beta actin promoter, ubiquitin promoter, JeT promoter. SV40 promoter, beta globin promoter, elongation Factor 1 alpha (EF1-alpha) promoter, Mo-MLV-LTR promoter, Rosa26 promoter, and any combination thereof.

In further embodiments, other elements to enhance transcription, translation, and/or selection, e.g., introns, polyadenylation sequences, marker sets, etc., can be present in the transgene constructs of the subject invention. The person with skill in the art can readily recognize the advantageous function of these elements and can readily include the respective elements in the constructs of the subject invention.

In some embodiments, the transgene is preferentially inserted into a chromosome at a transcriptionally active site. By way of example, transcriptionally active sites on Y chromosomes in bovine animals may include, but are not limited to, chromodomain Y like (CDY) genes, PRMAY, and members of the ZNF280BY and ZNF280AY autosome-derived Y chromosome gene families.

In some embodiments, the transgene is a selectable or screenable marker, such as a green fluorescent protein (GFP). Accordingly, in some embodiments, the circular polynucleotide of the present invention has the following elements from 5′ to 3′: sgRNA target site, left homology arm (match to cattle Y chromosome), FRT site, CMV enhancer, CMV promoter, Kozak sequence, GFP, SV40 poly(A) signal, FRT site, right homology arm (match to cattle Y chromosome), and sgRNA target site

Excision Sequences

In some embodiments, the new polynucleotide sequence further comprises a selectable or screenable marker optionally flanked by excision sequences.

In some embodiments, the excision sequences are IoxP sites or FRT sites. The Cre-IoxP and Flp-FRT systems are technologies that can be used to induce site-specific recombination events. Cre recombinase is an enzyme that removes DNA by homologous recombination between binding sequences known as lox-P sites. The Flp-FRT system operates in a similar way, with the Flip recombinase recognizing FRT sequences.

Various site-specific recombination systems such as Cre-loxP, Flp-FRT. Gateway (Invitrogen), ParA-res, and TnpR-res may be used in the methods of the present invention. These site-specific recombination systems may be useful for removal of a selectable or screenable marker. In conventional genome targeting, targeted clones are selected for using a resistance marker or a fluorescent protein; however, it is often desirable to remove the marker after the initial selection process. By way of example, by placing LoxP sites on both sides of the marker, the Cre recombinase can catalyze excision of the marker.

Genomic Deletion

In some aspects, the present invention may be useful in deleting a sequence in a genome. Accordingly, in some embodiments, the first targeting homologous region and the second targeting homologous region on the circular polynucleotide lack a third genome region between them, which is between the first genome homologous region and the second genome homologous region. After completion of genome-editing using the methods of the present invention, the third genome region is deleted as a result of homologous recombination.

The third genome region for deletion may be of a variety of lengths. In some embodiments, the third genome region is less than 9000, 8000, 7000, 6000, 5,000, 4,000, 3,000, 2,500, 2,000, 1,500, 1,000, 900, 800, 700, 600, 500, 400, 300, 250, 200, 150, 100, 90, 80, 70, 60, 50, 40, 30, 25, 20, 18, 16, or 14 nucleotides long. In some embodiments, the third genome region is at least 10, 12, 14, 16, 18, 20, 25, 30, 35, 40, 45, 50, 60, 70, 80, 90, 100, 125, 150, 175, 200, 225, 250, 300, 350, 400, 500, 600, 700, 800, 900, 1,000, 1,250, 1,500, 1,750, 2,000, 2,250, 2,500, 2,750, 3,000, 3,250, 3,500, 3,750, 4.000, 4,250, 4,500, 4,750, 5,000, 6,000, 7,000, 8,000, or 9,000 nucleotides long.

### Compositions and Kits for Genome-Editing

In certain aspects, the present invention provides a genome-edited cell produced by any one of the methods of the present invention.

In certain aspects, the present invention provides stabilized crRNAs, tracrRNAs, guide RNAs (gRNAs) or single guide RNAs (sgRNAs).

In certain aspects, the present invention provides a composition comprising the genome-editing polypeptide, the circular polynucleotide, and optionally the cell of any one of the preceding methods.

### Genome-Edited Animals

In some embodiments that may be combined with any one of the preceding embodiments, the methods of the invention further comprise transferring the genome-edited cell into a suitable host female animal to produce a genome-edited animal.

Accordingly, in certain aspects, the present invention provides a genome-edited animal produced by any one of the methods of the present invention.

Any technology known in the art appropriate for producing non-human genome-edited or transgenic animals may be used to practice the subject invention. Techniques for producing non-human genome-edited or transgenic animals are well known in the art and include, but are not limited to, pronuclear microinjection, viral infection, and transformation of embryonic stem cells and induced pluripotent stem (iPS) cells. Detailed methods that can be used include, but are not limited to, those described in Sundberg and Ichiki (2006, Genetically Engineered Mice Handbook, CRC Press). Hofker and van Deursen (2002, Genetically modified Mouse Methods and Protocols. Humana Press). Joyner (2000. Gene Targeting: A Practical Approach, Oxford University Press), Turksen (2002, Embryonic stem cells: Methods and Protocols in Methods Mol Biol., Humana Press), Meyer et al. (2010, Proc. Nat. Acad. Sci. USA 107:15022-15026), and Gibson (2004, A Primer Of Genome Science 2nd ed. Sunderland. Mass.: Sinauer), U.S. Pat. No. 6,586,251, Rathinam et al. (2011, Blood 118:3119-28), Willinger et al., (2011. Proc Natl Acad Sci USA, 108:2390-2395). Rongvaux et al., (2011. Proc Natl Acad Sci USA, 108:2378-83) and Valenzuela et al (2003, Nat Biot 21:652-659). Accordingly, in some embodiments, the method of the present invention further comprises transferring into a suitable host female animal the zygote, the embryo, a zygote or an embryo produced from the gamete, or an embryo produced from the zygote, optionally after screening for introduction of the new polypeptide into the genome of the cell or for deletion of the third genome region from the genome of the cell.

The animals in the methods of the subject invention can include any suitable animals including, but not limited to, such as bovids, equids, ovids, canids, cervids, felids, goats, swine, primates as well as less commonly known mammals such as elephants, deer, zebra, camels, or kudu. This list of animals is intended to be exemplary of the great variety of animals from which a cell can be routinely obtained. In some embodiments of the present invention, the animal is a bovine, an equine, a caprine, an ovine, a canine, a cervid, or a porcine animal.

### EXAMPLES

### Example 1: High Efficiency Homologous Repair-Based Gene Editing in Cattle

This example describes high efficiency homology-mediated end joining (HMEJ)-based gene editing in cattle using CRISPR/Cas9

### Materials and Methods

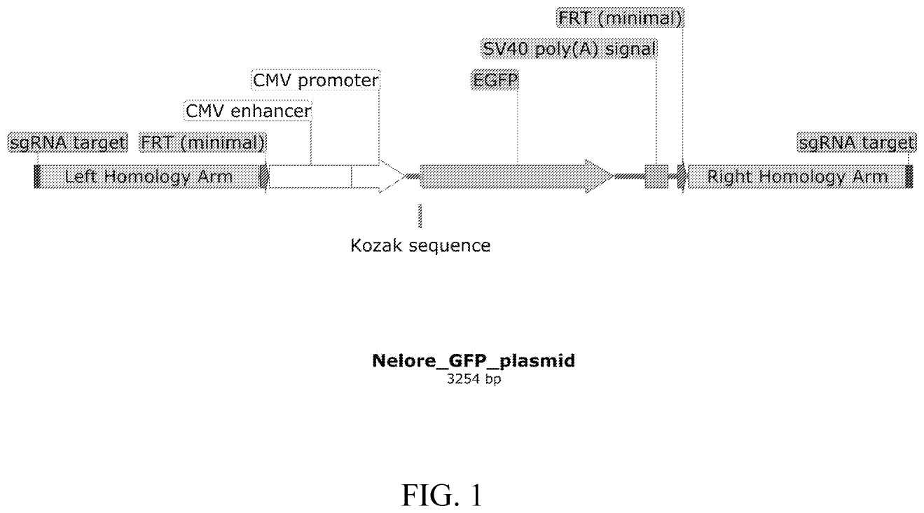

The gene editing materials used in this example included an HMEJ genetic construct, a purified Cas9 protein, and a purified sgRNA. The HMEJ genetic construct was assembled with a pUC57 backbone and was injected into cattle oocytes as described below as a circular plasmid, which can be excised from the plasmid using the same sgRNA that cleaves the insertion site on the cattle Y chromosome. FIG. 1 illustrates the structure of the genetic construct. SEQ ID NO. 1 shows the nucleotide sequence of the genetic construct. The purified Cas9 protein and purified sgRNA were commercially sourced. The sgRNA has a sequence of CACTGTGCACATTCTCCTAC (SEQ ID NO. 2) attached to the canonical Cas9 bound sequence, which matches the targeted Y chromosome site and is included at both ends of the HMEJ plasmid, allowing CRISPR/Cas9 excision of the recombination construct from the plasmid after injection into embryos.

The evening prior to injection, cattle oocytes were placed in maturation media M199, which contains LH/FSH/E2/ITS and 10% FBS with Gentamicin and incubated in 5% C02, air 02, at 37° C. overnight. Insemination was done via fertilization media and sp-talp. A half straw of semen was thawed and centrifuged in a 1.5 ml Eppendorf with 500 μl 50% percoli and 500 μl 25% percoli, 15 min at 300 g, 2× rinse in SP-TALP and the resultant pellet was resuspended in 100 μl fertilization media. Oocytes were placed in 80 μl drops of fertilization media in groups of no more than 30. Semen was added, 10-20 μl per drop depending on concentration.

Three hours prior to injection, two 4 well plates were prepared with 500 μl C4 media (5% CO2, 5% 02) for equilibration. Plates were prepared for denudation as follows: 100×15 plates had rows of drops consisting of 80 μl B0 media, 20 μl 1% Hyaluronidase solution in B0 media, and covered (to prevent evaporation) with 25 ml mineral oil. These were placed on a heater plate. Just prior to injection, holding and enucleation/transfer needles were placed on micromanipulators perpendicular to each-other with the tips in a single drop of PVP 10% until use.

Denudation occurred at 6-8 hours post-fertilization. Denudation time points later than 8-hr post fertilization were found to result in higher chimerism rates, as was use of Cas9 mRNA instead of Cas9 protein. Using a P10 tip, fertilized bovine embryos were moved in groups of 25 in the denudation media plates established above. With a p200, each fertilized egg was aspirated 10 times set at 100 μl followed by gentle aspiration with a borosilicate glass pipette with a 200 μM opening until the fertilized egg is denuded.

Microinjection was accomplished as follows, approximately 1 hour after denudation. Media mix was prepared in 10 μl aliquots, consisting of 1 μl Cas9 protein at 100 ng/μl, 1 μl sgRNA at 50 ng/μl, and 8 μl HMEJ plasmid at 800 ng/μl and was kept at −20 C until use. Ultra-pure water was used to control for effects of injection. The top of a 100 mm dish was prepared with 2 rows of 6 50 μl drops of B0 media with 0.2% PVP final, and covered with 25 ml fisher light mineral oil. Needles with rinsed with 20 μl drops of 10% PVP, 2 μl of premixed injection media (above) were loaded onto the plate just prior to microinjection as follows. Using a fire-polished pulled capillary tube attached to a p10 with a filtered tip, 2 μl of the thawed media mix were aspirated and placed on the plate. The needles were moved into the top row of 2 drops and rinsed well to establish good flow control. During the injection, the room was cooled to 18° C. Then 25 denuded embryos were placed singly into the microinjection drops established above. The ICSI pipette was loaded with injection media slowly over the course of 3 minutes, and the syringe advanced to a slow positive flow. After verification in 10% PVP, each embryo was injected quickly with a single piczo pulse to break the oolemma and injection a small (approximately 4 picoliters) bolus of media.

After injection, groups were placed in C4 media prepared three hours previously (described above), labeled, and additional groups completed. Once all groups were completed, they were placed in a fresh 700 μl well of C4 media (maximum of 100 embryos/well). On day 4, the cleaved (8 cell and higher) embryos were transferred to C5 media. Embryo development and gene integration was evaluated on day 8.

### Results

Table 1 shows results of the four rounds of oocyte injections. Overall cleavage rate was around 40%, similar for both control and the CRISPR injections. This is about half of the historical rates for in vitro fertilization (IVF) that average about 80%, which was likely due to the damage to the oocytes during the injection procedure. However, after the cleavage stage, the percentage of embryos continuing to develop matches the industry standard: roughly 40% of cleaved embryos reached the blastocyst stage, which is fairly typical. These results suggest that by day 2-3, any damage from the injection process has been resolved.

TABLE 1
Results of oocyte injections.

| Round | Group | Oocytes | Cleavage | Blastocysts | GFP+ | GFP− | % GFP+ | % Cl. | % Bl. | Bl./Cl. |
|---|---|---|---|---|---|---|---|---|---|---|
| 1 | CRISPR | 62 | 32 | 18 | 8 | 10 | 44% | 52% | 29% | 56% |
| 2 | CRISPR | 68 | 32 | 12 | 7 | 5 | 58% | 47% | 18% | 38% |
| 3 | CRISPR | 50 | 21 | 5 | 3 | 2 | 60% | 42% | 10% | 24% |
| 4 | CRISPR | 117 | 30 | 7 | 3 | 4 | 43% | 26% | 6% | 23% |
| | Total | 297 | 115 | 42 | 21 | 21 | 50% | 39% | 14% | 37% |
| 2 | Control | 23 | 13 | 8 | 0 | 8 | 0% | 57% | 35% | 62% |
| 4 | Control | 29 | 1 | 1 | 0 | 1 | 0% | 3% | 3% | 100% |
| | Total | 52 | 14 | 9 | 0 | 9 | 0% | 27% | 17% | 64% |

No GFP was visible in any cell at day 2-3, but by day 5, half of injected embryos expressed GFP (FIG. 1), which persisted through Day 8 when embryos were frozen for molecular analyses. Because the construct that was used is only capable of integrating into Y chromosome embryos, and because half of oocytes would have randomly been fertilized with Y chromosome-bearing sperm (the semen used for IVF was not sexed), the observed 50% embryos showing expressed GFP is the maximum percentage feasible (half of the embryos were female, for which correct integration was not possible).

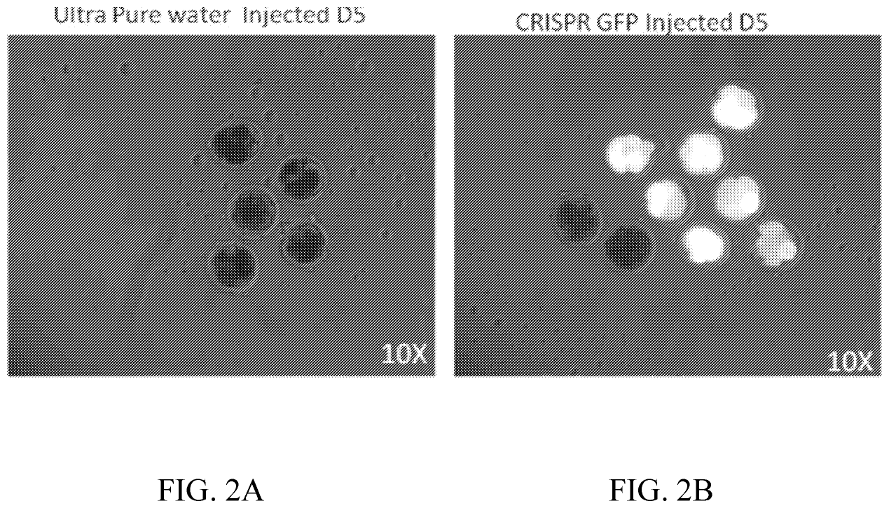

By Day 8, injected embryos fell into three categories—roughly half still expressed no GFP, about ⅙ expressed GFP in some, but not all, cells in the embryo (at this point consisting of several hundred cells), and the remaining ⅓ expressed GFP in apparently every cell in the embryo (FIG. 2).

Chimerism/Mosaicism. or lack thereof, was examined by extracting DNA from the embryos using a commercial low cell number DNA kit and performing PCR with primers amplifying: (a) an autosomal location AngII, to verify the extracted DNA; (b) a Y-chromosome site distant from the insertion site, to determine sex of the embryo; (c) a site indicating GFP integration into the Y chromosome; and (D) a region across the insertion site on the Y chromosome, which is only positive when the GFP is not integrated, all normalized to genomic beta actin primers (Table 2).

TABLE 2
Results of PCR verification.

| GFP | | | Y | Across | |
|---|---|---|---|---|---|
| Visible? | Tested | AngII | Chr. | Ins. | GFP |
| GFP− | 8 | 8 | 0 | 0 | 0 |
| Mosaic | 2 | 2 | 2 | 2 | 2 |
| GFP+ | 4 | 4 | 4 | 0 | 4 |

Results showed that firstly, of eight embryos selected with no visible GFP or integration, every embryo was female, indicating that the large majority of embryos with Y chromosome were successfully edited, at least with chimerism. Similarly, every embryo expressing GFP was male. Secondly, both visibly mosaic/chimeric and visibly non-chimeric embryos expressing GFP showed correct site-specific integration of GFP. Finally, while the chimeric embryos continued to contain DNA in which the integration site had not been interrupted, the visibly non-chimeric embryos were completely negative for an intact insertion site, indicating that no cells in those embryos did not contain GFP.

Taken together, this data shows that the method of invention surprisingly produces an extremely high rate of integration of the exogenous construct into the correct site in cattle: those that were negative were all female, indicating that very few males (likely <10%) lack modification. The visible chimerism rate was about ⅓ of those with any integration, however, ⅔ of the embryos had no chimerism, and presence of chimerism is testable. It is recognized that the number of tested samples in this experiment may be too low to assess the mosaicism rate confidently, but the sum of the data shows that efficiency of the genome-editing process of the present invention is very high and with few off-target integrations. The genome-editing process of invention would be very similar and may be used in all Artiodactyla (even-toed ungulates), including, without limitation, pigs, sheep, goats, and cattle.

This example thus demonstrates that a robust method of site-specific integration was successfully established with a relatively low chimerism rate, based on co-injection of CRISPR-nuclease with guide RNA that both cleave a specific site in the Y chromosome, as well as excising the homologous recombination construct from the plasmid. It is noted that although Cas9 protein was used in this example, any functionally similar protein from any of the many Cas9 variants (e.g. Cpf1) would work with the present invention. Ideally, second- or third-generation CRISPR approaches may be used, which, by using single-stranded rather than double-stranded nucleases, would reduce off-target effects.

### Example 2: High Efficiency Homologous Repair-Based Gene Editing in Goats

This example describes high efficiency homology-mediated end joining (HMEJ)-based gene editing in goats using CRISPR/Cas9.

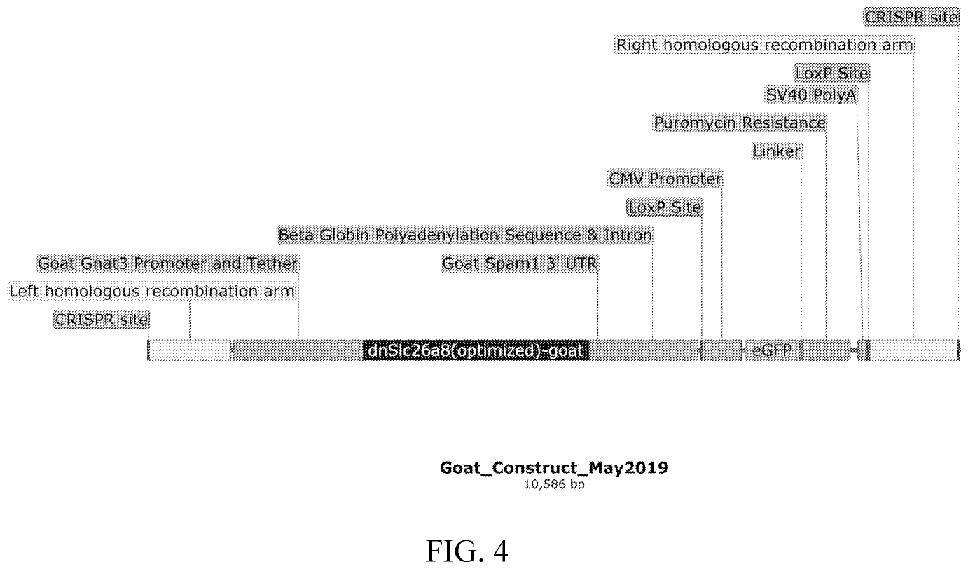

The gene editing materials used in this example included an HMEJ genetic construct, a purified Cas9 protein, and a purified sgRNA. The HMEJ genetic construct was assembled with a pUC57 backbone and was injected into goat primary fibroblasts as a circular plasmid, which can be excised from the plasmid using the same sgRNA that cleaves the insertion site on the goat Y chromosome. The key feature of this genetic construct is the presence of CRISPR sites in the circular plasmid which match the CRISPR site in between the left and right homology arms, such that the linear recombination construct is cut from the plasmid simultaneously with cleavage of the target integration site. FIG. 4 illustrates the structure of the genetic construct. SEQ ID NO. 3 shows the nucleotide sequence of the genetic construct. The purified Cas9 protein and purified sgRNA were commercially sourced. The sgRNA has a sequence of ACCAAAGTGATTATGGCTGA (SEQ ID NO. 4) attached to the canonical Cas9 bound sequence, which matches the targeted Y chromosome site and is included at both ends of the HMEJ plasmid, allowing CRISPR/Cas9 excision of the recombination construct from the plasmid after injection into embryos.

Cas9 protein, sgRNA, and HMEJ construct were transfected through normal methods into goat primary fibroblast cells, and the resulting cells were checked for correct integration. Cells were found to have a high rate of correct integration by examining expression of GFP and assay of PCR, with the first 12 single-cell subclones picked all possessing correct integration.

Taken together, this data shows that the method of invention surprisingly produces an extremely high rate of integration of the exogenous construct into the correct site in goats. It is noted that although Cas9 protein was used in this example, any functionally similar protein from any of the many Cas9 variants (e.g. Cpf1) would work with the present invention. Ideally, 2^{nd} or 3^{rd} generation CRISPR approaches would be used, which, by using single-stranded rather than double-stranded nucleases, would reduce off-target effects.

### Example 3: High Efficiency Homologous Repair-Based Gene Editing Using TALEN

This example describes high efficiency homology-mediated end joining (HMEJ)-based gene editing in an animal (e.g. cattle, goat, sheep, and pig) using transcription activator-like effector nuclease (TALEN).

The gene editing materials in this example includes an HM FJ genetic construct and a purified TALEN protein. The HMEJ genetic construct is assembled with a pUC57 backbone and is injected as a circular plasmid. The TALEN protein has a DNA binding domain that recognize a target sequence on the Y chromosome, and this target sequence is also included at both ends of the HMEJ plasmid, allowing TALEN excision of the recombination construct from the plasmid after injection into embryos. Accordingly, the HMEJ construct comprises from 5′ to 3′ the following elements: TALEN target site, left homology arm, FRT site, CMV enhancer, CMV promoter, Kozak sequence, GFP, SV40 poly(A) signal. FRT site, right homology arm, and TALEN target site. The key feature of this genetic construct is the presence of TALEN target sites in the circular plasmid which match the TALEN target site in between the left and right homology arms, such that the linear recombination construct is cut from the plasmid simultaneously with cleavage of the target integration site.

TALEN protein and the HMEJ construct are transfected through normal methods into animal embryos, and the resulting embryos are checked for correct integration by examining expression of GFP and assay of PCR.

It is noted that editing with TALEN is likely to produce fewer or no off-target cleavage sites as compared to CRISPR, but use of TALEN would likely have a lower efficiency. To overcome the lower efficiency, a few cells would be extracted from day 8 embryos, and the remainder of the embryo frozen using standard methodologies. The cells extracted would be expanded, to allow pre-implantation assays for correct integration (e.g., sequencing across the insertion site to show full, correct integration into the correct site).

### Example 4: High Efficiency Homologous Repair-Based Gene Editing Using ZFN

This example describes high efficiency homology-mediated end joining (HMEJ)-based gene editing in an animal (e.g. cattle, goat, sheep, and pig) using (zinc finger nuclease) ZFN.

The gene editing materials in this example includes an HMEJ genetic construct and a purified ZFN protein. The HMEJ genetic construct is assembled with a pUC57 backbone and is injected as a circular plasmid. The ZFN protein has a DNA binding domain that recognize a target sequence on the Y chromosome, and this target sequence is also included at both ends of the HMEJ plasmid, allowing ZFN excision of the recombination construct from the plasmid after injection into embryos. Accordingly, the HMEJ construct comprises from 5′ to 3′ the following elements: ZFN target site, left homology arm, FRT site, CMV enhancer, CMV promoter, Kozak sequence. GFP, SV40 poly(A) signal, FRT site, right homology arm, and ZFN target site. The key feature of this genetic construct is the presence of ZFN target sites in the circular plasmid which match the ZFN target site in between the left and right homology arms, such that the linear recombination construct is cut from the plasmid simultaneously with cleavage of the target integration site.

ZFN protein and the HMEJ construct are transfected through normal methods into animal embryos, and the resulting embryos are checked for correct integration by examining expression of GFP and assay of PCR.

It is noted that editing with ZFN is likely to produce fewer or no off-target cleavage sites as compared to CRISPR, but the use of ZFN would likely have a lower efficiency. To overcome the lower efficiency, a few cells would be extracted from day 8 embryos, and the remainder of the embryo frozen using standard methodologies. The cells extracted would be expanded, to allow pre-implantation assays for correct integration (e.g., sequencing across the insertion site to show full, correct integration into the correct site).

### Example 5: High Efficiency Homologous Repair-Based Gene Editing in Pigs

This example describes high efficiency homology-mediated end joining (HMEJ)-based gene editing in pigs using CRISPR/Cas9 or CRISPR/Cas12.

The gene editing materials for use in this example include an HMEJ genetic construct, a purified Cas9 or a purified Cas12, and a purified sgRNA. The HMEJ genetic construct is assembled with a pUC57 backbone and is injected into pig primary fibroblasts as a circular plasmid, which can be excised from the plasmid using the same sgRNA that cleaves the insertion site on the pig Y chromosome. The key feature of this genetic construct is the presence of CRISPR sites in the circular plasmid, which match the CRISPR site in between the left and right homology arms, such that the linear recombination construct is cut from the plasmid simultaneously with cleavage of the target integration site. The purified Cas9 protein and purified sgRNA may be commercially sourced. The sgRNA has a sequence of attached to the canonical Cas9 or Cas12 bound sequence, which matches the targeted Y chromosome site and is included at both ends of the HMEJ plasmid, allowing CRISPR/Cas9 or CRISPR/Cas12 excision of the recombination construct from the plasmid after injection into embryos.

Cas9 or Cas12 protein, sgRNA, and HMEJ construct is transfected through normal methods into pig primary fibroblast cells, and the resulting cells are checked for correct integration. Cells will be found to have a high rate of correct integration by examining expression of GFP and assay of PCR.

Taken together, this example will show that the method of invention surprisingly produces an extremely high rate of integration of the exogenous construct into the correct site in pigs. It is noted that although Cas9 or Cas12 protein is used in this example, any functionally similar protein from any of the many Cas9 or Cas12 variants would work with the present invention. Ideally, 2^{nd} or 3^{rd} generation CRISPR approaches would be used, which, by using single-stranded rather than double-stranded nucleases, would reduce off-target effects.

### Example 6: High Efficiency Homologous Repair-Based Gene Editing in Sheep

This example describes high efficiency homology-mediated end joining (HMEJ)-based gene editing in sheep using CRISPR/Cas9 or CRISPR/Cas12.

The gene editing materials for use in this example include an HMEJ genetic construct, a purified Cas9 or a purified Cas12, and a purified sgRNA. The HMEJ genetic construct is assembled with a pUC57 backbone and is injected into sheep primary fibroblasts as a circular plasmid, which can be excised from the plasmid using the same sgRNA that cleaves the insertion site on the sheep Y chromosome ne key feature of this genetic construct is the presence of CRISPR sites in the circular plasmid, which match the CRISPR site in between the left and right homology arms, such that the linear recombination construct is cut from the plasmid simultaneously with cleavage of the target integration site. The purified Cas9 protein and purified sgRNA may be commercially sourced. The sgRNA has a sequence of attached to the canonical Cas9 or Cas12 bound sequence, which matches the targeted Y chromosome site and is included at both ends of the HMEJ plasmid, allowing CRISPR/Cas9 or CRISPR/Cas12 excision of the recombination construct from the plasmid after injection into embryos.

Cas9 or Cas12 protein, sgRNA, and HMEJ construct is transfected through normal methods into sheep primary fibroblast cells, and the resulting cells are checked for correct integration. Cells will be found to have a high rate of correct integration by examining expression of GFP and assay of PCR.

Taken together, this example will show that the method of invention surprisingly produces an extremely high rate of integration of the exogenous construct into the correct site in sheep. It is noted that although Cas9 or Cas12 protein is used in this example, any functionally similar protein from any of the many Cas9 or Cas12 variants would work with the present invention. Ideally, 2^{nd} or 3^{rd} generation CRISPR approaches would be used, which, by using single-stranded rather than double-stranded nucleases, would reduce off-target effects.

1: A method for high efficiency homologous repair based genome-editing comprising:
(a) providing a cell from a bovine, an equine, a caprine, an ovine, a cervid, or a porcine animal, wherein the cell comprises a genome comprising a first genome homologous region, a second genome homologous region, and a genome cut site between the first genome homologous region and the second genome homologous region; and
(b) introducing a genome-editing polypeptide that introduces at least a single stranded break at the genome cut site and a circular polynucleotide comprising a first targeting homologous region and a second targeting homologous region, wherein either (i) the circular polynucleotide comprises a new polynucleotide sequence between the first targeting homologous region and the second targeting homologous region, or (ii) the first targeting homologous region and the second targeting homologous region lack a third genome region between them that is between the first genome homologous region and the second genome homologous region,
wherein the first targeting homologous region is homologous to the first genome homologous region and the second targeting homologous region is homologous to the second genome homologous region, and
wherein the genome-editing polypeptide introduces a least one strand break at the genome cut site and either (1) the new polynucleotide sequence is introduced into the genome of the cell between the first genome homologous region and the second genome homologous region by homologous recombination between the first genome homologous region and the first targeting homologous region and between the second genome homologous region and the second targeting homologous region, or (2) the third genome region is deleted from the genome of the cell by homologous recombination between the first genome homologous region and the first targeting homologous region and between the second genome homologous region and the second targeting homologous region.

2: The method of claim 1, wherein the circular polynucleotide further comprises a first circular polynucleotide cut site 5′ to the first targeting homologous region and optionally a second circular polynucleotide cut site 3′ to the second targeting homologous region.

3: The method of claim 1, wherein the cell is an induced pluripotent stem cell (iPSC), an embryonic stem cell (ESC), a progenitor of a gamete, a gamete, a zygote, or a cell in an embryo.

4: The method of claim 3, further comprising transferring into a suitable host female animal the zygote, the embryo, a zygote or an embryo produced from the gamete, or an embryo produced from the zygote, optionally after screening for introduction of the new polypeptide into the genome of the cell or for deletion of the third genome region from the genome of the cell.

5: The method of claim 2, wherein the genome-editing polypeptide introduces at least a single stranded break at the first circular polynucleotide cut site, the second circular polynucleotide cut site, or both.

6: The method of claim 2, wherein a second genome-editing polypeptide is introduced to the cell sequentially or simultaneously with the genome-editing polypeptide and the second genome-editing polypeptide introduces at least a single stranded break at the first circular polynucleotide cut site, the second circular polynucleotide cut site, or both.

7: The method of claim 1, wherein the genome-editing polypeptide is a site-specific nuclease polypeptide, a TALEN polypeptide or a ZFN polypeptide.

8. (canceled)

9: The method of claim 1, wherein the genome-editing polypeptide is a CRISPR-nuclease polypeptide in complex with a targeting polynucleotide that hybridizes at or adjacent to the genome cut site, and optionally the CRISPR-nuclease polypeptide is a single-strand-specific or double-strand-specific nuclease that is site-directed by a guide RNA or the CRISPR-nuclease polypeptide is a Cas9 polypeptide, a Cas12 polypeptide, a Cascade polypeptide, or a CasZ polypeptide.

10-11. (canceled)

12: The method of claim 1, wherein:
(a) the genome-editing polypeptide introduces a double-stranded break that is blunt or staggered;
(b) the circular polynucleotide is a vector or a plasmid;
(c) the circular polynucleotide does not comprise a bacterial origin of replication;
(d) the new polynucleotide sequence comprises one or more point mutations; and/or
(e) the new polynucleotide sequence comprises a transgene.

13-15. (canceled)

16: The method of claim 12, wherein:
(a) the one or more point mutations introduce a stop codon in a polypeptide coding region at or adjacent to the genome cut site; introduce a new DNA-binding site for a transcription enhancer or a transcription repressor at or adjacent to the genome cut site; alter or eliminate a DNA-binding site for a transcription enhancer or a transcription repressor at or adjacent to the genome cut site; or change a gene at or adjacent to the genome cut site from a first allele to a second allele;
(b the one or more point mutations is at least 2, 3, 4, 5, 6, 7, 8, 9, 10 15, 20, 25, 30, 35, 40, or 50 insertions, deletions, substitutions, or combinations thereof; and/or
(c) the one or more point mutations is less than 2, 3, 4, 5, 6, 7, 8, 9, 10 15, 20, 25, 30, 35, 40, 50, 60, 70, 80, 90, or 100 insertions, deletions, substitutions, or combinations thereof.

17-19. (canceled)

20: The method of claim 12, wherein the transgene comprises one or more of the following: a promoter region; an enhancer region; a transcription termination regions, and a polypeptide coding region that optionally further comprises a polyadenylation site.

21: The method of claim 1, wherein the new polynucleotide sequence further comprises a selectable or screenable marker optionally flanked by excision sequences, and optionally the excision sequences are loxP sites or FRT sites.

22. (canceled)

23: The method of claim 1, wherein the third genome region is less than 9000, 8000, 7000, 6000, 5,000, 4,000, 3,000, 2,500, 2,000, 1,500, 1,000, 900, 800, 700, 600, 500, 400, 300, 250, 200, 150, 100, 90, 80, 70, 60, 50, 40, 30, 25, 20, 18, 16, or 14 nucleotides long and/or the third genome region is at least 10, 12, 14, 16, 18, 20, 25, 30, 35, 40, 45, 50, 60, 70, 80, 90, 100, 125, 150, 175, 200, 225, 250, 300, 350, 400, 500, 600, 700, 800, 900, 1,000, 1,250, 1,500, 1,750, 2,000, 2,250, 2,500, 2,750, 3,000, 3,250, 3,500, 3,750, 4,000, 4,250, 4,500, 4,750, 5,000, 6,000, 7,000, 8,000, or 9,000 nucleotides long.

24. (canceled)

25: The method of claim 9, wherein adjacent to the genome cut site is within 5,000, 4,000, 3,000, 2,500, 2,000, 1,500, 1,000, 900, 800, 700, 600, 500, 400, 300, 250, 200, 150, 100, 90, 80, 70, 60, 50, 40, 30, 20, 10, or 5 nucleotides of the at least single stranded break.

26: The method of claim 1, wherein:
(a) the first genome homologous region is less than 5,000, 4,000, 3,000, 2,500, 2,000, 1,500, 1,000, 900, 800, 700, 600, 500, 400, 300, 250, 200, 150, 100, 90, 80, 70, 60, 50, 40, 30, 25, 20, 18, 16, or 14 nucleotides long;
(b) the first genome homologous region is at least 10, 12, 14, 16, 18, 20, 25, 30, 35, 40, 45, 50, 60, 70, 80, 90, 100, 125, 150, 175, 200, 225, 250, 300, 350, 400, 500, 600, 700, 800, 900, 1,000, 1,250, or 1,500 nucleotides long;
(c) the second genome homologous region is less than 5,000, 4,000, 3,000, 2,500, 2,000, 1,500, 1,000, 900, 800, 700, 600, 500, 400, 300, 250, 200, 150, 100, 90, 80, 70, 60, 50, 40, 30, 25, 20, 18, 16, or 14 nucleotides long; and/or
the second genome homologous region is at least 10, 12, 14, 16, 18, 20, 25, 30, 35, 40, 45, 50, 60, 70, 80, 90, 100, 125, 150, 175, 200, 225, 250, 300, 350, 400, 500, 600, 700, 800, 900, 1,000, 1,250, or 1,500 nucleotides long.

27-29. (canceled)

30: The method of claim 1, wherein the first genome homologous region and the second genome homologous region are on the same chromosome.

31: The method of claim 1, wherein:
(a) the first genome homologous region and the second genome homologous region are less than 9000, 8000, 7000, 6000, 5,000, 4,000, 3,000, 2,500, 2,000, 1,500, 1,000, 900, 800, 700, 600, 500, 400, 300, 250, 200, 150, 100, 90, 80, 70, 60, 50, 40, 30, 25, 20, 18, 16, or 14 nucleotides apart;
(b) the first genome homologous region and the second genome homologous region are at least 10, 12, 14, 16, 18, 20, 25, 30, 35, 40, 45, 50, 60, 70, 80, 90, 100, 125, 150, 175, 200, 225, 250, 300, 350, 400, 500,600, 700, 800, 900, 1,000, 1,250, 1,500, 1,750, 2,000, 2,250, 2,500, 2,750, 3,000, 3,250, 3,500, 3,750, 4,000, 4,250, 4,500, 4,750, 5,000, 6,000, 7,000, 8,000, or 9,000 nucleotides apart;
(c) the new polynucleotide sequence is at least 10, 12, 14, 16, 18, 20, 25, 30, 35, 40, 45, 50, 60, 70, 80, 90, 100, 125, 150, 175, 200, 225, 250, 300, 350, 400, 500, 600, 700, 800, 900, 1,000, 1,250, 1,500, 1,750, 2,000, 2,250, 2,500, 2,750, 3,000, 3,250, 3,500, 3,750, 4,000, 4,250, 4,500, 4,750, 5,000, 6,000, 7,000, 8,000, 9,000, 10,000, 12,500, 15,000, or 20,000 nucleotides long;
(d) the new polynucleotide sequence is less than 50, 60, 70, 80, 90, 100, 125, 150, 175, 200, 225, 250, 300, 350, 400, 500, 600, 700, 800, 900, 1,000, 1,250, 1,500, 1,750, 2,000, 2,250, 2,500, 2,750, 3,000, 3,250, 3,500, 3,750, 4,000, 4,250, 4,500, 4,750, 5,000, 6,000, 7,000, 8,000, 9,000, 10,000, 12,500, 15,000, 20,000, 30,000, 40,000, or 50,000 nucleotides long; and/or
(e) the new polynucleotide sequence is between 50 and 50,000, 60 and 50,000, 70 and 50,000, 80 and 50,000, 90 and 50,000, 100 and 50,000, 125 and 50,000, 150 and 50,000, 175 and 50,000, 200 and 50,000, 225 and 50,000, 250 and 50,000, 300 and 50,000, 350 and 50,000, 400 and 50,000, 500 and 50,000, 600 and 50,000, 700 and 50,000, 800 and 50,000, 900 and 50,000, 1,000 and 50,000, 1,250 and 50,000, 1,500 and 50,000, 1,750 and 50,000, 2,000 and 50,000, 2,250 and 50,000, 2,500 and 50,000, 2,750, 3,000 and 50,000, 3,250 and 50,000, 3,500 and 50,000, 3,750 and 50,000, 4,000 and 50,000, 4,250 and 50,000, 4,500 and 50,000, 4,750 and 50,000, 5,000 and 50,000, 6,000 and 50,000, 7,000 and 50,000, 8,000 and 50,000, 9,000 and 50,000, 10,000 and 50,000, 12,500 and 50,000, 15,000 and 50,000, or 20,000 and 50,000 nucleotides long.

32-35. (canceled)

36: A composition comprising the genome-editing polypeptide, the circular polynucleotide, and optionally the cell of the method of claim 1.

37: A genome-editing kit comprising the genome-editing polypeptide, the circular polynucleotide, and optionally the cell of the method of claim 1.

38: A genome-edited animal produced by the method of claim 4.

39-41. (canceled)
